(19) United States
(12) Patent Application Publication
(10) Pub. No.: US 2022/0054615 A1
(43) Pub. Date: Feb. 24, 2022

(54) PERTUSSIS BOOSTER VACCINE

(71) Applicant: SANOFI PASTEUR INC., Swiftwater, PA (US)

(72) Inventors: Nicolas BURDIN, Marcy L'Etoile (FR); Martina OCHS, Marcy L'Etoile (FR); Marie GARINOT, Marcy L'Etoile (FR); Martine CHABAUD-RIOU, Marcy L'Etoile (FR); Nathalie REVENEAU, Marcy L'Etoile (FR); Yuanqing LIU, Marcy L'Etoile (FR); Noelle MISTRETTA, Marcy L'Etoile (FR)

(21) Appl. No.: 17/299,244

(22) PCT Filed: Nov. 29, 2019
(86) PCT No.: PCT/US19/63840

§ 371 Date: Jun. 2, 2021

(30) Foreign Application Priority Data
Dec. 5, 2018 (EP) 18306621.6

(51) Int. Cl. A61K 39/02; A61K 39/05; A61K 39/08; A61K 39/39; A61P 31/04

(52) U.S. Cl. CPC A61K 39/099; A61K 39/05; A61K 2039/55511; A61K 39/39; A61P 31/04; A61K 39/08

(57) ABSTRACT

The present disclosure is directed to a modified acellular pertussis booster vaccine comprising a TLR agonist and methods of using the same for inducing an immune response.

### CROSS REFERENCE TO RELATED APPLICATIONS

This application claims the benefit of, and relies on the filing date of, European Patent Application No. 18306621.6, filed 5 Dec. 2018, the entire contents of which are incorporated herein by reference.

### SEQUENCE LISTING

The instant application contains a Sequence Listing which has been filed electronically in ASCII format and is hereby incorporated by reference in its entirety. Said ASCII copy, created on Nov. 27, 2019, is named 0171_0016-PCT SL and is 1 kilobyte in size.

### FIELD

The present disclosure relates to acellular pertussis vaccines and methods of using the same.

### BACKGROUND

Pertussis, or whooping cough is an acute and highly contagious respiratory disease caused primarily by Bordetella pertussis. Before the broad implementation of immunization programs, pertussis was highly endemic. Evidence suggests that almost all children became infected with B. pertussis before they reached adulthood, with most of them suffering some degree of clinical disease, and that high circulation of the bacterium provided natural boosting of infection-acquired immunity, which estimates suggest lasted from 7-10 to 20 years (Wendelboe et al., Pediatr Infect Dis J, 2005; 24: S58-S61).

Vaccination has been the most effective strategy to reduce the number of cases of pertussis (Halperin, N Engl J Med. 2005; 353:1615-7). The initial pertussis vaccines included killed whole cells of B. pertussis (wP) that were chemically detoxified and formulated with diphtheria and tetanus antigens. Since the 1990s, wP vaccines have been replaced in many countries by acellular pertussis vaccines. Acellular pertussis (aP) vaccines induce relatively fewer side-effects compared to wP vaccines, which are associated with a high risk for fever, reactogenicity at the injection site and, to a lesser extent, convulsions. Current acellular vaccines are typically based on the following virulence factors: pertussis toxin (PT), filamentous hemagglutinin (FHA), pertactin (PRN), fimbrial agglutinogen 2 and fimbrial agglutinogen 3 (FTM2/3 or FIM). While some acellular vaccines contain only PT and FHA or PT alone, it is generally believed that acellular pertussis vaccines containing PT, FHA, PRN, and FIM2/3 components are the most effective aP vaccines currently available.

Notwithstanding decades of vaccination, whooping cough remains an endemic disease worldwide with locally specific epidemic peaks or outbreaks occurring every 2 to 5 years (typically 3 or 4 years), without a consistent seasonal pattern (Edwards et al., Whooping Cough Vaccine. In Vaccines 6th edition. Edited by Plotkin S, Orenstein W, Offit P. 6th ed. Philadelphia, Elsevier, 2012:447-92; JD Cherry, Pediatrics 2005; 115:1422-27). Despite large differences in the reported incidence between countries, the highest age-specific incidence of pertussis cases, hospitalizations and complications is now reported in infants <1 year of age and mainly before 3 months of age in each country. Infants too young to have completed their primary vaccine series account for the majority of pertussis-related complications, hospitalizations, and deaths (Bisgard et al., Pediatr Infect Dis J. 2004; 23:985-89; Haberling et al., Pediatr Infect Dis J. 2009; 28(3):194-98; Public Health England, Health Protection Report. 2014; 8(17); Centers for Disease Control and Prevention, MMWR 2012; 61(28):517-22; Winter et al., J Pediatr. 2012; 161:1091-96). In recent epidemiologic observations, adolescents and adults tend to exhibit the second highest incidence of disease and highest increase in incidence in countries with well-established vaccination schedules, such as the United States or the United Kingdom.

The accumulated evidence suggests that the observed resurgence and outbreaks likely results from the combined effects of multiple factors including awareness (Kaczmarek et al., MJA 2013; 198:624-8), increased sensitivity of laboratory confirmation methods (Tarr et al., Am J Epidemiol. 2013; 178(2):309-18), incomplete schedules or suboptimal vaccine coverage (Atwell et al., Pediatrics 2013; 132:624-30; Quinn et al., Pediatrics 2014; 133(3):e513-9; Glanz et al., JAMA Pediatr. 2013; 167(11); 1060-64; Imdad et al., Pediatrics 2013; 132:37-43), waning and change in nature of vaccine-induced immunity, along with genotypic and phenotypic changes in the organism (Lam et al., Australia Emerg Infect Dis. 2014; 20:626-33; (Pawloski et al., Clin Vaccine Immunol. 2014; 21(2):119-25; Martin et al., Clin Infect Dis. 2014; 60(2):223-27).

To reduce the incidence of pertussis in children, aP booster vaccinations around the age of 4-6 have been implemented in many countries (Zepp et al., Lancet Infect Dis, 2011; 11(7):557-70; Clark et al., NASN Sch Nurse, 2012; 27(6):297-300). The efficacy of booster doses of acellular pertussis-containing vaccines (Tdap) has been evaluated in several settings. One randomized clinical trial conducted in the United States between 1997 and 1999 evaluated the efficacy of a Tdap vaccine in adolescents and adults aged 15 through 65. This study found that Tdap vaccination was associated with a decreased incidence of clinical illness (i.e., cough greater than 21 days) and of increases in anti-pertussis antibody levels over the 1-year observation period, for a vaccine efficacy estimate of 92% against laboratory-confirmed pertussis (Ward et al., Clin Infect Dis 2006; 43:151-57). The effectiveness of Tdap vaccination in pertussis control was also shown in several observational studies in non-outbreak settings in the US and Australia, demonstrating a significant impact of adolescent Tdap vaccination on disease incidence; one study estimated an effectiveness of 85.4% against laboratory confirmed pertussis (Skoff et al., Arch Pediatr Adolesc Med. 2012; 166(4):344-49; Rank et al., Pediatr Infect Dis J. 2009; 28(2):152-53; Quinn et al., Bull World Health Organ 2011; 89:666-674). However, several recent case-control studies conducted in US outbreak settings in cohorts that were mostly primed with acellular vaccines consistently estimated a moderate effectiveness for Tdap vaccination around 65% or lower against laboratory-confirmed pertussis (Acosta et al., Washington State, 2012. IDWeek 2013 Meeting of the Infectious Diseases Society of America. Poster 139; Wei et al., Clinical Infectious Diseases 2010; 51(3):315-21; Baxter et al., BMJ. 2013; 347:f4249; Liko et al., N Engl J Med. 2013 Feb. 7; 368(6):581-82; Klein et al., Pediatrics. 2016; 137(3):e20153326). Despite the inherent, methodological limitations present in most of these observational studies, the evidence of rapid waning of all-brand effectiveness following the Tdap booster dose observed in the Wisconsin study was consistent with the observations of the study conducted in the Washington state outbreak setting, which estimated that the effectiveness of Tdap vaccination waned rapidly from 75% in the first year following the booster dose to 42% from the second year onward (Koepke et al., J Infect Dis. 2014; 210(6); 942-53; Acosta et al., IDWeek 2013 Meeting of the Infectious Diseases Society of America. Poster 139). The epidemiologic trends and distribution of pertussis cases in the US and in Canada over recent years have been interpreted as corroborating the concept that aP vaccine-primed individuals responded less robustly to pertussis booster vaccines and that their protection waned faster than in previous cohorts primed with wP vaccines (A. Acosta, Advisory Committee on Immunization Practices. Summary Report June 19-20, 2013; Chambers et al., CCDR. 2014; 40(3):31-41). In addition, several studies analyzing data obtained in the US outbreaks in 2010 and 2012 and in the 2008-2012 Australian outbreaks suggest that the protection elicited by booster vaccines wanes faster in aP vaccine-primed than in wP vaccine-primed individuals (Liko et al., N Engl J Med. 2013 Feb. 7; 368(6):581-82; Smallridge et al., Infect Dis. 2014; 209(12):1981-88; Witt et al., Clin Infect Dis. 2012; 54(12):1730-35; Klein et al., N Engl J Med. 2012; 367(11):1012-19; Tartof et al., Pediatrics. 2013; 131(4):e1047-52; Sheridan et al., JAMA. 2012; 308(5):454-56; Witt et al., Clin Infect Dis. 2013; May; 56(9):1248-54; Klein et al., Pediatrics. 2013; 131(6):e1716-22). Although the results of these studies have been challenged by some experts on methodological bases, their conclusions were supported by the ecologic observations reported by the CDC in the US, showing waning of protection occurring sooner in the aP-primed cohorts compared to previous observations in wP primed cohorts (A. Acosta, Advisory Committee on Immunization Practices. Summary Report June 19-20, 2013). In fact, the comparison of immunogenicity results obtained in clinical studies involving aP or wP vaccine-primed adolescents provides consistent evidence with lower humoral and cellular (B and Th1) immune responses one-month post-Tdap vaccine administration (Marshall et al., Clinical and Vaccine Immunology. 2014; 21(11):1560-64; Sanofi Pasteur Clinical Trial Td516, Final Clinical Statistical Report, Version 1.0 dated 11 Jun. 2010; Sanofi Pasteur Clinical Trial Td551, Final Clinical Study Report, Version 1.0 dated 16 Jul. 2013; van der Lee et al., Front Immunol. 2018; 9:51).

While a modeling study based on the Swedish pertussis surveillance data suggested a herd effect in pertussis epidemiology in the aP vaccine context, several other studies using surveillance data from the US, Italy and other countries have found that the model that best suits the observed epidemiologic trends tend to be the ones which hypothesize that aP vaccines elicit lower booster response, faster waning of protection and higher asymptomatic transmission than wP vaccines (Althouse et al, BMC Med. 2015; 13(1):146-57; Domenech et al., Proc Natl Acad Sci USA. 2014; 111(7):E716-7; Magpantay et al., Parasitology 2016; 143:835-49).

A possibly different protective mechanism for wP and aP vaccines has been suggested, based on a study by Warfel et al. in non-human primates (Warfel et al., Proc Natl Acad Sci USA. 2014; 111:787-92). Although this study was not in humans and was in a non-statistically significant sample, it found that aP vaccines prevented clinical disease but did not clear colonization as quickly as wP vaccines and permitted transmission to susceptible animals (Id.). Similar conclusions were also reached from a study using a mouse model of pertussis (Smallridge et al., J Infect Dis. 2014; 209(12):1981-88). The studies conducted in non-human primates in particular pointed to a differential immunological profile following wP vaccination compared to aP priming. Specifically, a Th1 or mixed Th1/Th17 response profile was associated with wP vaccine priming while a Th2 profile was associated with aP vaccine priming. It has consequently been hypothesized that the Th1/Th17 bias of the immune response observed after wP vaccination may be associated with longer duration of protection and faster clearance of infection (i.e., stronger initial protection) than with the Th2 bias observed after aP vaccination. Further, it has been observed that the initial Th1/Th17 versus Th2 responses induced by primary vaccination with wP and aP, respectively, are maintained following aP booster vaccination, even years after the initial primary vaccination, suggesting that the priming vaccination influences the Th1/Th17 versus Th2 bias following aP booster vaccination (Bancroft et al., Cell Immunol, 2016, 304-305:35-43). The key role of Th1/Th17 effector cells in immunity against B. pertussis has been confirmed in the murine model of infection, further strengthening this hypothesis (Ross et al., PLoS Pathog. 2013; 9(4): e1003264). While human evidence supporting this hypothesis has yet to be developed, some research has suggested differences in the T helper cell responses in people who received wP versus aP vaccines (Fedele G. et al., Pathog Dis 2015; 73(7): doi: 10.1093/femspd/ftv051). In addition, the Th2 dominated response to the aP vaccines also appears to correspond with a different IgG isotype distribution. The response to aP vaccination produces mainly IgG1, but also IgG2 and IgG4 with the proportion of IgG4 increasing after booster vaccinations. In contrast, the Th1 dominated response of wP vaccines is accompanied predominantly by IgG1 and IgG2 antibody responses (Brummelman et al., Pathog Dis. 2015; 73(8); Diavatopoulos et al., Cold Spring Harb Perspect Biol. 2017 Mar. 13; van der Lee et al., Vaccine 2018; 36(2):220-26).

It would be beneficial to provide improved booster aP vaccines having longer lasting protection against B. pertussis infection.

### SUMMARY

The present inventors have developed new, modified acellular pertussis (aP) booster vaccines comprising a toll-like receptor (TLR) agonist. More specifically, the present inventors surprisingly found that administering an aP booster vaccine with a TLR4 and/or a TLR9 agonist can reorient a Th2-biased immune response, induced by a previously administered aP vaccine, towards a Th1-biased immune response. Without intending to be bound by any theory, it appears that the repolarization of T helper cells and shift in Th1/Th2 balance induced by the modified aP booster vaccine is associated with accelerated B. pertussis clearance.

A first aspect of this disclosure is directed to an aP booster vaccine, comprising a tetanus toxoid, a diphtheria toxoid, a detoxified pertussis toxin (typically a genetically-modified pertussis toxin), filamentous hemagglutinin, pertactin, fimbriae types 2 and 3, a TLR agonist, and an aluminum salt, wherein at least the TLR agonist is formulated with an aluminum salt (Aspect 1).

Another aspect is directed to a method of inducing an immune response in a human subject who has previously been exposed to B. pertussis antigens, the method comprising administering to the subject an aP booster vaccine, wherein the aP booster vaccine comprises a tetanus toxoid, a diphtheria toxoid, a detoxified pertussis toxin, filamentous hemagglutinin, pertactin, fimbriae types 2 and 3, at least one TLR agonist, and an aluminum salt, wherein at least the TLR agonist is formulated with an aluminum salt, and wherein administering the aP booster vaccine reorients a Th2-biased immune response, induced by previous exposure to B. pertussis antigens, towards a Th1-biased immune response or a Th1/Th17-biased immune response in the human subject (Aspect 2). Also covered by Aspect 2 is the use of the aP booster vaccine for reorienting a Th2-biased immune response toward a Th1-biased or Th1/Th17-biased immune response in a human subject who has been previously exposed to B. pertussis antigens, typically via an aP priming vaccine.

Typically, in the context of Aspect 2, the human subject has previously received an acellular pertussis vaccine (also referred to herein as an aP priming vaccine) prior to administering the aP booster vaccine, which aP priming vaccine induces a Th2-biased immune response. Alternatively, the human subject may have been previously exposed to B. pertussis antigens by receiving a wP vaccine or through a natural infection with B. pertussis. Typically, when a human subject who has received an aP priming vaccine receives an aP booster vaccine that does not contain a TLR agonist (e.g., ADACEL®), the non-TLR agonist containing aP booster vaccine boosts the Th2-biased immune response induced by the aP priming vaccine. By contrast, the modified, TLR agonist containing aP booster vaccine described herein unexpectedly reorients the Th2-biased immune response induced by the aP priming vaccine towards a Th1-biased immune response or a Th1/Th17-biased immune response.

In certain embodiments of Aspect 2, the Th1-biased immune response is characterized by one or more of decreased IL-5 production, increased IFN-γ production, or a lower IgG1/IgG2a ratio, as compared to the immune response induced by the aP priming vaccine or an aP booster vaccine that does not contain a TLR agonist (e.g., ADACEL®). In certain embodiments of Aspect 2, the Th1-biased immune response is characterized by decreased IL-5 production and/or a lower IgG1/IgG2a ratio, as compared to the immune response induced by the aP priming vaccine or an aP booster vaccine that does not contain a TLR agonist (e.g., ADACEL®). In certain embodiments of Aspect 2, a Th1/Th17-biased response is characterized by increased IL-17 production and one or more of decreased IL-5 production or a lower IgG1/IgG2a ratio. In certain embodiments of Aspect 2, a Th1/Th17-biased response is characterized by increased IL-17 production and one or more of decreased IL-5 production or a lower IgG1/IgG2a ratio, as compared to the immune response induced by the aP priming vaccine or an aP booster vaccine that does not contain a TLR agonist (e.g., ADACEL®).

In certain embodiments of Aspects 1 and 2, the TLR agonist is a TLR4 agonist. Preferably, the TLR4 agonist is an agonist of human TLR4. In certain embodiments, the TLR4 agonist is E6020.

In other embodiments of Aspects 1 and 2, the TLR agonist is a TLR9 agonist. Preferably, the TLR9 agonist is an agonist of human TLR9. In certain embodiments, the TLR9 agonist is a CpG oligonucleotide. In certain embodiments, the CpG oligonucleotide is a Class A, Class B, Class C, or Class P CpG oligonucleotide. In certain embodiments, the CpG oligonucleotide is CpG1018, having the nucleotide sequence of SEQ ID NO: 1 in which all the nucleotides in SEQ ID NO: 1 are linked with a phosphorothioate linkage.

In certain embodiments of Aspects 1 and 2, the tetanus toxoid is present in an amount of 8-12 Lf/mL, optionally 9-11 Lf/mL, or optionally 10 Lf/mL.

In certain embodiments of Aspects 1 and 2, the diphtheria toxoid is present in an amount of 3-8 Lf/mL, optionally, 3-6 Lf/mL, or optionally 4-5 Lf/mL.

In certain embodiments of Aspects 1 and 2, the detoxified pertussis toxin is a genetically detoxified pertussis toxin (gdPT). In certain embodiments, the gdPT comprises a mutation at R9. In certain embodiments, the gdPT comprises a R9K mutation and an E129G mutation. In certain embodiments, the gdPT is present in an amount of 4-30 μg/mL, optionally 16-24 μg/mL, optionally 18-22 μg/mL, or optionally 20 μg/mL.

In certain embodiments of Aspects 1 and 2, the filamentous hemagglutinin is present in an amount of 5-15 μg/mL, optionally 8-12 μg/mL, or optionally 10 μg/mL.

In certain embodiments of Aspects 1 and 2, the pertactin is present in an amount of 5-15 μg/mL, optionally 8-12 μg/mL, or optionally 10 μg/mL.

In certain embodiments of Aspects 1 and 2, the fimbriae types 2 and 3 are present in an amount of 10-20 μg/mL, optionally 14-16 μg/mL or optionally 15 μg/mL.

In certain embodiments of Aspects 1 and 2, the TLR4 agonist, such as E6020, is present in an amount of no more than 10 μg/mL, optionally 0.5-5 μg/mL, or optionally no more than 2 μg/mL.

In certain embodiments of Aspects 1 and 2, the TLR9 agonist, such as CpG1018, is present in an amount of 250-750 μg/mL, optionally 400-600 μg/mL, or optionally 500 μg/mL.

In certain embodiments of Aspects 1 and 2, the aP booster vaccine comprises tetanus toxoid in an amount of about 8-12 Lf/mL and optionally 9-11 Lf/mL; diphtheria toxoid in an amount of about 3-8 Lf/mL and optionally 3-6 Lf/mL; genetically-detoxified pertussis toxin in an amount of about 16-24 μg/mL and optionally 18-22 μg/mL; filamentous hemagglutinin in an amount of about 5-15 μg/mL and optionally 8-12 μg/mL; pertactin in an amount of about 5-15 μg/mL and optionally 8-12 μg/mL; fimbriae types 2 and 3 in an amount from about 10-20 μg/mL and optionally 14-16 μg/mL; aluminum hydroxide (A100H) in an amount of about 0.25-0.75 mg/mL and optionally 0.6-0.7 mg/mL; and a TLR4 agonist, such as E6020, in an amount of no more than 10 μg/mL and optionally 0.5-5 μg/mL or optionally 1-2 μg/mL. Alternatively, in place of the TLR4 agonist, the aP booster vaccine may contain a TLR9 agonist, such as CpG1018, in an amount of 250-750 μg/mL and optionally 400-600 μg/mL.

In certain embodiments of Aspects 1 and 2, the aP booster vaccine comprises tetanus toxoid in an amount of 10 Lf/mL; diphtheria toxoid in an amount of 4-5 Lf/mL; genetically-detoxified pertussis toxin in an amount of 20 μg/mL; filamentous hemagglutinin in an amount of 10 μg/mL; pertactin in an amount of 10 μg/mL; fimbriae types 2 and 3 in an amount 15 μg/mL; aluminum hydroxide (A100H) in an amount of 0.66 mg/mL; and a TLR4 agonist, such as E6020, in an amount of no more than 2 μg/mL. Alternatively, in place of the TLR4 agonist, the aP booster vaccine may contain a TLR9 agonist, such as CpG1018, in an amount of 500 μg/mL.

In certain embodiments of Aspects 1 and 2, the aP booster vaccine is in a unit dose form for administration to a human subject and comprises tetanus toxoid in an amount of 4-6 Lf, optionally 4.5-5.5 Lf, or optionally 5 Lf per 0.5 mL dose.

In certain embodiments of Aspects 1 and 2, the aP booster vaccine is in a unit dose form for administration to a human subject and comprises diphtheria toxoid in an amount of 1-4 Lf, optionally 1.5-3 Lf, or optionally 2-2.5 Lf.

In certain embodiments of Aspects 1 and 2, the aP booster vaccine is in a unit dose form for administration to a human subject which is typically per 0.5 mL dose and comprises genetically-detoxified pertussis toxin in an amount of 2-12 μg, optionally 8-12 μg, or optionally 10 μg.

In certain embodiments of Aspects 1 and 2, the aP booster vaccine is in a unit dose form for administration to a human subject and comprises filamentous hemagglutinin in an amount of 2.5-7.5 ng, optionally 4-6 μg, or optionally 5 μg.

In certain embodiments of Aspects 1 and 2, the aP booster vaccine is in a unit dose form for administration to a human subject and comprises pertactin is present in an amount of 2.5-7.5 μg, optionally 4-6 μg, or optionally 5 μg.

In certain embodiments of Aspects 1 and 2, the aP booster vaccine is in a unit dose form for administration to a human subject and comprises fimbriae types 2 and 3 in an amount of 5-10 μg, optionally 7-8 μg or optionally 7.5 μg.

In certain embodiments of Aspects 1 and 2, the aP booster vaccine is in a unit dose form for administration to a human subject and comprises the TLR4 agonist, such as E6020, in an amount of no more than 5 μg, optionally 0.25-2.5 μg, or optionally no more than 1 μg.

In certain embodiments of Aspects 1 and 2, the aP booster vaccine is in a unit dose form for administration to a human subject and comprises the TLR9 agonist, such as CpG1018, in an amount of 125-375 μg, optionally 200-300 μg, or optionally 250 μg.

In certain embodiments of Aspects 1 and 2, the aP booster vaccine is in a unit dose form for administration to a human subject and comprises the following components per 0.5 mL dose: tetanus toxoid in an amount of about 4-6 Lf and optionally 4.5-5.5 Lf; diphtheria toxoid in an amount of about 1-4 Lf and optionally 1.5-3 Lf; genetically-detoxified pertussis toxin in an amount of about 2-12 μg and optionally 8-12 μg; filamentous hemagglutinin in an amount of about 2.5-7.5 μg and optionally 4-6 μg; pertactin in an amount of about 2.5-7.5 μg and optionally 4-6 μg, fimbriae types 2 and 3 in an amount of about 5-10 μg and optionally 7-8 μg; aluminum hydroxide (A100H) in an amount of about 0.125-0.375 mg and optionally 0.3-0.35 mg; and a TL4 agonist, such as E6020, in an amount of no more than 5 μg and optionally 0.25-2.5 μg. Alternatively, in place of the TLR4 agonist, the aP booster vaccine may contain a TLR9 agonist, such as CpG1018, in an amount of 125-375 μg and optionally 200-300 μg.

In certain embodiments of Aspects 1 and 2, the aP booster vaccine is in a unit dose form for administration to a human subject and comprises the following components per 0.5 mL dose: tetanus toxoid in an amount of 5 Lf, diphtheria toxoid in an amount of 2-3 Lf, genetically-detoxified pertussis toxin in an amount of 10 μg, filamentous hemagglutinin in an amount of 5 μg, pertactin in an amount of 5 fimbriae types 2 and 3 in an amount from about 7.5 μg, aluminum hydroxide (A100H) in an amount of 0.33 mg, and a TLR4 agonist, such as E6020, in an amount of no more than 1 μg. Alternatively, in place of the TLR4 agonist, the aP booster vaccine may contain a TLR9 agonist, such as CpG1018, in an amount of 250 μg.

In certain embodiments of Aspects 1 and 2, the aP booster vaccine further comprises one or more of the following antigens: Haemophilus influenzae type-b oligosaccharide or polysaccharide conjugate (Hib), hepatitis B virus surface antigen (HBsAg) and/or inactivated polio virus types 1, 2 and 3 (IPV).

In certain embodiments of Aspects 1 and 2, the aP booster vaccine further comprises a tris-buffered saline.

In certain embodiments of Aspects 1 and 2, the aP booster vaccine has an aluminum concentration of 0.25-0.75 mg/mL, optionally 0.6-0.7 mg/mL, or optionally 0.66 mg/mL. In certain embodiments of Aspects 1 and 2, the aP booster vaccine is in a unit dose form for administration to a human subject and contains aluminum in an amount of 0.125-0.375 mg, optionally 0.3-0.35 mg, or optionally 0.33 mg.

In certain embodiments of Aspects 1 and 2, at least one of the tetanus toxoid, the diphtheria toxoid, and the detoxified pertussis toxin is adsorbed to the aluminum salt. In certain embodiments, the tetanus toxoid, the diphtheria toxoid, the detoxified pertussis toxin, filamentous hemagglutinin, pertactin, and fimbriae types 2 and 3 are adsorbed to the aluminum salt. In certain embodiments, the TLR4 agonist or TLR9 agonist is formulated with the aluminum salt. In certain embodiments, all of the bacterial antigens in the aP booster vaccine and the TLR4 or TLR9 agonist are formulated with the aluminum salt.

In certain embodiments of Aspects 1 and 2, the aluminum salt is an aluminum hydroxide. In certain embodiments of Aspects 1 and 2, the aluminum salt is an aluminum phosphate.

In certain embodiments of Aspects 1 and 2, the aP booster vaccine further comprises a Haemophilus influenzae type-b saccharide (Hib) conjugate, a hepatitis B virus surface antigen (HBsAg) and/or an inactivated polio virus (IPV).

In certain embodiments of Aspect 2, the human subject is 4 years of age or older when the aP booster vaccine is administered.

In certain embodiments of Aspect 2, the human subject is 10 years of age or older when the aP booster vaccine is administered.

In certain embodiments of Aspect 2, the Th1-biased immune response is characterized by one or more of decreased IL-5 production, increased IFN-γ production, increased IL-17 production, or a lower IgG1/IgG2a ratio, as compared to the acellular pertussis vaccine or an aP booster vaccine that does not contain a TLR agonist (e.g., ADACEL®). In certain embodiments, the Th1-biased immune response is characterized by one or more of decreased IL-5 production or a lower IgG1/IgG2a ratio, as compared to the acellular pertussis vaccine or an aP booster vaccine that does not contain a TLR agonist (e.g., ADACEL®).

In certain embodiments of Aspect 2, the aP priming vaccine comprises a tetanus toxoid, a diphtheria toxoid, a detoxified pertussis toxin, filamentous hemagglutinin, and pertactin, with the proviso that the aP priming vaccine does not contain a TLR agonist. In certain embodiments, the aP priming vaccine includes but is not limited to one or more doses of DAPTACEL®, INFANRIX®, INFANRIX-HEXA®, PENTACEL®, QUADRACEL®, KINRIX®, PEDIARIX®, or VAXELIS®. In certain embodiments, the aP priming vaccine comprises DAPTACEL®. In certain embodiments, the aP priming vaccine comprises INFANRIX® or INFANRIX-HEXA®. In certain embodiments, the aP priming vaccine comprises PENTACEL®. In certain embodiments, the aP priming vaccine comprises KINRIX® or PEDIARIX®. In certain embodiments, the aP priming vaccine comprises VAXELIS®.

Further areas of applicability of the present disclosure will become apparent from the detailed description provided hereinafter. It should be understood that the detailed description and specific examples, while indicating some desirable aspects of the disclosure, are intended for purposes of illustration only and are not intended to limit the scope of the disclosure.

### BRIEF DESCRIPTION OF THE FIGURES

FIGS. 1A-B show a comparison of genetically-detoxified PT (gdPT) immunogenicity with chemically-detoxified PT (PTxd) at eliciting a PT specific antibody response. FIG. 1A shows Anti-PT, IgG1 and IgG2a antibody titers measured by ELISA after 2 immunizations. FIG. 1B shows Anti-PT neutralizing titers as measured by CHO assay after 2 immunizations. Mice were immunized two times at day 0 and 21. Mice were bled 17 days after the second immunization (Day 38).

FIGS. 2A-B show a comparison of the boostability of a PTxd-primed response with PTxd or gdPT. FIG. 2A shows the ELISA PT-specific IgG1 and IgG2a response after priming with PTxd or gdPT followed by boosting with PTxd or gdPT. FIG. 2B shows the PT neutralizing titers after priming with PTxd or gdPT followed by boosting with PTxd or gdPT, as measured by CHO assay. Mice were immunized three times at day 0, 21 and 42. Mice were bled 17 days after the second immunization and 8 days after the third immunization.

FIG. 3 shows the evaluation of absence of immunological interference by gdPT with the IgG1 and IgG2a responses induced against FIM antigens after 2 immunizations.

FIGS. 4A-B show the evaluation of immunological interference by other Tdap antigens on gdPT-induced anti-PT IgG1 and IgG2a responses and neutralizing antibody responses against PT antigens after 3 immunizations. FIG. 4A shows Anti-PT, IgG1 and IgG2a antibody titers as measured by ELISA after 3 immunizations. FIG. 4B shows Anti-PT neutralizing titers as measured by CHO assay after 3 immunizations.

FIG. 5 shows the ability of the modified Tdap (gdPT+E6020-A100H) and the modified Tdap (gdPT+CpG1018-A100H) formulations to down-modulate a DTaP-induced Th2 immune memory response using a long prime-boost schedule, by measuring cytokine (IL-5, IFN-γ, and IL-17) levels. Cytokine levels were measured by Fluorospot assay.

FIGS. 6A-L show the levels of different antigen-specific IgG1 and IgG2a induced in mice after different prime/boost schedules. Naïve adult CD1 mice were immunized (primed) i.m. with DTwP or DTaP. Forty-two days later (D42), DTwP-primed mice were boosted with DTwP vaccine and DTaP-primed mice were boosted with modified TdaP vaccines (mTdaP-A100H, mTdaP-CpG-A100H or mTdaP-E6020-A100H). FHA- and FIM2,3-, PRN-, PT-, DT-, and TT-specific IgG1 and IgG2a antibody responses were assessed by a modified ELISA technique (MSD) in sera collected 42 days post boost (D84) and again at 42 days later (D126) after a second boost at D84 using a long prime-boost schedule. The numbers above the IgG1 and IgG2a bars in FIGS. 6A-L represent the IgG1/IgG2a ratio with lower numbers representing lower ratios and vice versa. Lower IgG1/IgG2a ratios were observed in mice boosted with modified Tdap vaccines containing a TLR agonist (CpG or E6020). FIGS. 6A and 6B show the IgG1 and IgG2a results and ratios for FHA at D84 and D126 respectively. FIGS. 6C and 6D show the IgG1 and IgG2a results and ratios for FIM at D84 and D126 respectively. FIGS. 6E and 6F show the IgG1 and IgG2a results and ratios for PRN at D84 and D126 respectively. FIGS. 6G and 6H show the IgG1 and IgG2a results and ratios for gdPT at D84 and D126 respectively. FIGS. 6I and 6J show the IgG1 and IgG2a results and ratios for DT at D84 and D126 respectively. FIGS. 6K and 6L show the IgG1 and IgG2a results and ratios for TT at D84 and D126 respectively.

FIG. 7 depicts the mouse adoptive transfer model used for evaluating the new Tdap boost vaccine formulations.

FIGS. 8A-B show the kinetics of PT-, PRN-, FHA- and FIM2,3-specific IgG antibody responses in the mouse adoptive transfer model, as measured by the mean Log₁₀ IgG titers of pooled sera (n=4, 6-7 mice per pool)±SEM. FIG. 8A shows the IgG responses for DTaP/Tdap (prime/boost), DTwP/Tdap (prime/boost), or DTwP/DTwP (prime/boost) at several time points after boost. FIG. 8B shows accelerated and higher anti-PT, PRN, FHA, and FIM2,3 IgG titers of the modified Tdap (gdPT+E6020-A100H) boost and the modified Tdap (gdPT+CpG1018-A100H) boost as compared to Tdap boost response in adoptive transferred mice, following a DTaP priming vaccine. In FIG. 8A, the P-value<0.05 is indicated as follows: *DTaP/Tdap (PRIME/BOOST) versus DTwP/Tdap (PRIME/BOOST); #DTaP/Tdap (PRIME/BOOST) versus DTwP/DTwP (PRIME/BOOST); ‡DTwP/Tdap (PRIME/BOOST) versus DTwP/DTwP (PRIME/BOOST).

FIGS. 9A-B show that boosting with the modified Tdap (gdPT+E6020-A100H) and the modified Tdap (gdPT+CpG1018-A100H) formulations provides early and/or accelerated bacterial clearance after intranasal challenge with B pertussis. FIG. 9A shows results of clearance after priming with DTaP or DTwP followed by a Tdap or DTwP boost.

FIG. 9B shows results of clearance after priming with DTaP followed by a Tdap, modified Tdap (gdPT+E6020-A100H) or modified Tdap (gdPT+CpG1018) boost. FIGS. 9A and B show the Log₁₀ number of CFUs per lung at the indicated time points for n=3-4 mice per group ±SEM. In FIG. 9B, the P-value<0.05 is indicated as follows: *DTaP/Tdap (PRIME/BOOST) vs DTaP/mTdap-CP G-AL 00H (PRIME/BOOST); § DTaP/Tdap (PRIME/BOOST) vs DTaP/mTdap-CPG-ALOOH (PRIME/BOOST); +DTaP/Tdap (PRIME/BOOST) vs mTdap-E6020-ALOOH (PRIME only); $ DTaP/Tdap (PRIME/BOOST) vs mTdap-CPG-ALOOH (PRIME only); #DTaP/mTdap-CPG-ALOOH (PRIME/BOOST) vs DTaP/mTdap-E6020-ALOOH (PRIME/BOOST). Individual mice tested (4 per group per time-point) Statistical test: 2-way ANOVA. The symbols indicate the time points where significance is seen.

FIGS. 10A-B depict a mouse intranasal challenge assay (INCA) using a short immunization schedule (FIG. 10A) and a long schedule (FIG. 10B).

FIG. 10C shows that DTaP/Tdap (prime/boost) and DTwP/DTwP (prime/boost) protect mice against Bordetella pertussis lung colonization following intranasal challenge in the INCA short model.

FIG. 11 shows that the modified Tdap (gdPT+E6020-A100H) booster significantly accelerates B. pertussis clearance as compared to the Tdap booster in the mouse intranasal challenge assay (INCA) using a short prime-boost schedule.

FIGS. 12A-B show that the modified Tdap (gdPT+E6020-A100H) and the modified Tdap (gdPT+CpG1018-A100H) booster vaccines are able to protect DTaP primed mice against disease and colonization of the lower respiratory tract using a long prime-boost schedule. FIG. 12A shows the reduction in all vaccinated groups 3 days post challenge with baseline bacterial load reached at day 7 for all treatment groups. FIG. 12B shows that all acellular pertussis dosing schedules had similar kinetics as the DTwP/DTwP (prime/boost) dosing schedule.

FIGS. 13A-B show that boosting a DTaP priming immunization with a modified Tdap (gdPT+E6020-A100H) formulation induces IL-17 production in an E6020 dose dependent manner, as measured by a fluorospot assay. The splenocytes from CD1 mice immunized with DTaP and boosted twice with mTdap-E6020-A100H (DO and D21) were isolated and re-stimulated in vitro with PTx (FIG. 13A) or a pool of pertussis antigens consisting of PTx, PRN, and FIM (FIG. 13B).

FIG. 14 shows the thermal profiles of modified Tdap formulations: mTdap (gdPT+A100H), mTdap (gdPT+E6020-A100H), and mTdap (gdPT+CpG-A100H), showing the first derivative of intrinsic fluorescence emission ratio (350 nm/330 nm). Thermal transition (Tm) of mTdap (gdPT+A100H) is 74.6° C.; mTdap (gdPT+E6020-A100H) is 74.2° C.; and mTdap (gdPT+CpG-A100H) is 77.0° C.

### DETAILED DESCRIPTION

The following description of various desirable aspect(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

As used herein, “aP” refers to an acellular B. pertussis vaccine. Current acellular B. pertussis vaccines are typically based on the following virulence factors: detoxified pertussis toxin (PT), filamentous hemagglutinin (FHA), pertactin (PRN), fimbrial agglutinogen 2 and fimbrial agglutinogen 3 (FIM2/3 or FIM). While some acellular pertussis vaccines contain only PT and FHA or PT, FHA and PRN, it is generally believed that acellular pertussis vaccines containing PT, FHA, PRN, and FIM2/3 components are the most effective aP vaccines currently available. Typically acellular pertussis vaccines are formulated with diphtheria toxoid and tetanus toxoid.

As used herein, “wP” refers to a whole cell B. pertussis vaccine. Typically, whole cell pertussis vaccines include whole cells of B. pertussis that have been chemically detoxified and formulated with diphtheria toxoid and tetanus toxoid.

As used herein, “DTwP” refers to a wP indicated in the prevention of diphtheria, tetanus and pertussis in infants as a first vaccination and in children as a booster. One example of a DTwP is to D.T.COQ/D.T.P., which was marketed by Sanofi Pasteur and contains diphtheria toxoid and tetanus toxoid, B. pertussis inactivated by heat in the presence of thiomersal, and aluminum phosphate.

As used herein, “DTaP” refers to an aP indicated for active immunization against diphtheria, tetanus and pertussis in infants and children. Typically, the DTaP is administered as a five-dose series in infants and children 6 weeks through 6 years of age or a four-series dose series in infants and children 6 weeks through 2-4 years of age. Examples of DTaP include, but are not limited to, DAPTACEL®, PENTACEL®, and INFANRIX® or INFANRIX-HEXA®. DAPTACEL®, for example, is marketed by Sanofi Pasteur and contains diphtheria toxoid and tetanus toxoid, the following acellular pertussis antigens: PT (chemically detoxified), FHA, PRN, and FIM2/3, as well as aluminum phosphate. Typically, the DTaP contains increased amounts of diphtheria toxoid and PT as compared to the Tdap.

As used herein, “Tdap” refers to an aP indicated for active booster immunization against tetanus, diphtheria and pertussis. Typically, the Tdap is administered as a single dose in individuals 10 years of age and older. Examples of Tdap include, but are not limited to, ADACEL® and BOOSTRIX®. ADACEL®, for example, is marketed by Sanofi Pasteur and contains diphtheria toxoid and tetanus toxoid and the following acellular pertussis antigens: PT (chemically detoxified), FHA. PRN, and FIM2/3, as well as aluminum phosphate. Typically, the Tdap contains reduced amounts of diphtheria toxoid and PT as compared to the DTaP.

As used herein, “modified Tdap” or “mTdap” refers to a modified version of a Tdap vaccine comprising diphtheria toxoid and tetanus toxoid and the following acellular pertussis antigens: genetically modified PT, ERA, PRN, and FIM2/3. The modified Tdap is different from Tdap at least because the modified Tdap contains a TLR agonist (for example a TLR4 agonist (e.g., E6020) or a TLR9 agonist CpG1018)). The modified Tdap also optionally contains genetically-detoxified PT (gdPT) instead of chemically-detoxified PT (PTdx) or aluminum hydroxide instead of aluminum phosphate. In certain embodiments, the mTdap contains a TLR agonist (for example a TLR4 agonist E6020) or a TLR9 agonist (e.g., CpG1018)), genetically detoxified PT (gdPT), and aluminum hydroxide.

As used herein, “booster” or “booster vaccine” refers to a vaccine administered following a priming vaccine. The booster vaccine contains antigens that were included in the priming vaccine so that the immune system has already been exposed to such antigens prior to administration of the booster vaccine.

As used herein, “priming vaccine” refers to one or more doses of a vaccine in a vaccination schedule that are administered before a booster vaccine and induce a primary immune response and immunological memory.

Th1/Th2 Immune Responses

CD4 T helper cell responses to antigens can be classified based on the cytokines they produce. Type 1 helper T cells (Th1) preferentially produce inflammatory cytokines, such as IFN-γ, IL-2, TNF-α, and TNF-0. Th1 cells activate macrophages and are typically associated with cell-mediated immune responses and phagocyte-dependent protective responses (e.g., opsonizing antibodies). Type 2 helper cells (Th2), on the other hand, preferentially produce cytokines, such as IL-4, IL-5, IL-10, and IL-13. Th2 cells activate B cells and are typically associated with antibody-mediated immune responses.

Studies in children have shown that a wP priming vaccine preferentially induces a Th1-biased response, whereas an aP priming vaccine preferentially induces a Th2-biased response. Ryan et al., Immunology, 1998, 93:1-10; Ausiello et al., Infect Immun, 1997, 65:2168-74. In addition to inducing distinct cytokine profiles, aP priming vaccines in mice induce predominantly IgG1 antibodies, but also IgG2 and IgG4, with the proportion of IgG4 increasing after booster vaccinations, reflective of a Th2-biased response (Stenger et al., Vaccine, 2010, 28:6637-46 and Brummelman et al., Vaccine, 2015, 33:1483-19) whereas wP priming vaccines in mice induce predominantly IgG2 antibodies, as well as IgG1 and IgG3, (Raeven et al., J Proteome Res, 2015, 14:2929-42), consistent with a Th1-biased response.

As disclosed herein, the modified aP booster vaccines described in this application are able to reorient a Th2-biased immune response induced by a previously administered aP vaccine towards a Th1- or mixed Th1/Th17-biased immune response. As reflected in the relevant literature, including the references cited in the preceding paragraph, one of skill in the art is able to measure cytokine profiles and antibody isotypes using conventional techniques to readily determine if an aP vaccine induces a Th1-biased or Th2-biased response. Typically, a Th2-biased immune response induced by an aP vaccine in mice is associated with increased IL-5 levels and/or an increased IgG1/IgG2a ratio, whereas a Th1-biased immune response is typically associated with one or more of decreased IL-5 levels, increased IFN-γ levels, or a reduced IgG1/IgG2a ratio. For example, the ability of an aP booster vaccine as described herein to reorient a Th2-biased immune response induced by a previously administered aP vaccine towards a Th1-biased immune response indicates a reduction in IL-5 levels and/or a reduction in the IgG1/IgG2a ratio as compared to the immune response induced by previously administered aP vaccine or an aP booster vaccine that does not contain a TLR agonist. The Th17 response is measured by the production of IL-17.

Tetanus Toxoid

The tetanus toxoid is produced from Clostridium tentani, a Gram-positive, rod-shaped, spore-forming, bacillus bacteria. Tetanus toxoid is a protein of about 150 kDa and consists of two subunits (about 100 kDa and about 50 kDa) linked by a sulfide bond. The tetanus toxoid is typically detoxified with formaldehyde and can be purified from culture filtrates using known methods, such as ammonium sulfate precipitation and/or chromatography techniques, as disclosed, for example, in WO 1996/025425. Clostridium tentani can be grown in any suitable growth medium, including, for example, Mueller-Miller casamino acid medium without beef heart infusion (Mueller et al., J Bacteriol, 1954, 67(3):271-277) or a Latham medium derived from bovine casein. The tetanus toxoid may also be inactivated by recombinant genetic means.

The amount of tetanus toxoid can be expressed as an “Lf” unit (i.e., limit of flocculation or flocculating unit), which is defined as the amount of toxoid that when mixed with one International Unit of antitoxin, produces an optimally flocculating mixture. See Module 1 of WHO's The immunological basis for immunization series (Galazka). The amount of tetanus toxoid in a composition can be readily determined by comparing the composition to a reference material calibrated with reference reagents in a flocculation assay.

Diphtheria Toxoid

The diphtheria toxoid is an ADP-ribosylating exotoxin produced by Corynebacterium diphtheriae, a Gram positive, non-sporing aerobic bacterium. Like tetanus toxoid, the diphtheria toxoid is detoxified, typically using formaldehyde, to yield a toxoid that is not toxic but is still antigenic. C. diphtheriae can be grown in any suitable growth medium, such as modified Mueller's growth medium (Stainer, D W, In: Manclark C R, editor, Proceedings of an informal consultation of the WHO requirements for diphtheria, tetanus, pertussis and combined vaccines, U.S. Public Health Service, Bethesda, Md. DHHS 91-1174, 1991, 7-11) or Fenton medium or Linggoud and Fenton medium, which may be supplemented with bovine extract. The diphtheria toxin can be purified using conventional techniques, such as ammonium sulfate fractionation and detoxified either before or after purification using standard techniques, such as formaldehyde treatment.

As with the tetanus toxoid, the amount of diphtheria toxoid can be expressed as an “Lf” unit. The amount of diphtheria toxoid in a composition can be readily determined by comparing the composition to a reference material calibrated with reference reagents in a flocculation assay.

Pertussis Toxin

Pertussis toxin (PT) is a secreted protein exotoxin and an important virulence factor produced exclusively by B. pertussis. Pertussis toxin is composed of five subunits, named S1, S2, S3, S4 (x2) and S5 that are encoded by five genes organized into an operon of approximately 3200 base pairs. Expression of the five genes is regulated by a promoter located upstream of the 51 encoding gene. Activation of the toxin promoter is under control of Bordetella virulence gene (bvg) system, which regulates not only the expression of pertussis toxin, but other known virulence factors such as FHA and PRN.

Pertussis toxin is a protein of about 105 kDa with an A/B configuration. The A domain, composed of the 51 subunit, is responsible for the ADP-ribosylating activity of the protein. It blocks the binding of G protein (guanine nucleotide-binding protein) to G protein-coupled receptor (GPCR) on the host cell membrane thus interfering with signal transduction and resulting in many of the biologic effects associated with PT activity such as histamine-sensitization, leukocytosis and alteration in insulin secretion. The B oligomer is a pentameric ring composed of subunits S2, S3, S4, and S5, associated in the ratio 1:1:2:1 and is responsible for the binding to the receptor on eukaryotic cells. It binds to various (but mostly unidentified) glycoconjugate molecules on the surface of target cells.

The pertussis toxin used in current acellular vaccines is typically chemically detoxified. In the aP booster vaccine described herein, the detoxified pertussis toxin is typically genetically modified to reduce enzymatic activity and/or toxicity. Many constructs containing genetic modifications of pertussis toxin have been engineered to reduce enzymatic activity and/or toxicity of the protein while preserving its immunogenicity and protective properties, including, for example, mutant pertussis toxin having a mutation at amino acid 129 of the S1 subunit of the pertussis toxin, such as the E129G mutant or the R9K/E129G double mutant. See e.g., U.S. Pat. Nos. 5,433,945, 7,144,576, 7,666,436, and 7,427,404. Thus, in certain embodiments, the genetically detoxified pertussis toxin contains a mutation at amino acid 129 of the S1 subunit of pertussis toxin. In certain embodiments, the mutation is an E129G mutation. In certain embodiments, the genetically detoxified pertussis toxin contains an R9K mutation and an E129G. Although a genetically detoxified pertussis toxin is preferred, it is also possible to use a chemically detoxified pertussis toxin in place of the genetically detoxified pertussis toxin. Chemical detoxification can, for instance, be performed by any of a variety of conventional chemical detoxification methods, such as treatment with formaldehyde, hydrogen peroxide, tetranitromethane, or glutaraldehyde. See e.g., U.S. Pat. No. 5,877,298.

Pertactin

Pertactin is a 69 kDa outer membrane protein originally identified from B. bronchiseptica (Montaraz, J. A. et al. Infect. Immun. 1985; 161:581-582). It was shown to be a protective antigen against B. bronchiseptica and was subsequently identified in both B. pertussis and B. parapertussis. The 69 kDa protein binds directly to eukaryotic cells (Leininger, E. et al., Proc Natl Acad Sci USA 1991; 88:345-349) and natural infection with B. pertussis induces an anti-pertactin humoral response (Thomas, M. G. et al. J. Infect. Dis. 1989; 159:21148). Pertactin also induces a cell-mediated immune response (Petersen, al., Infect. Immun. 1992; 60:4563-70; De Magistris, T. et al., J. Exp. Med. 1988; 168:1351-1362; Seddon, et al., Serodiagnosis Immunother. Inf. Dis. 1990; 3:337-43). Vaccination with whole-cell or acellular vaccines induces anti-pertactin antibodies (Edwards, K. M. et al., Pediatr. Res. 1992; 31:91A; Podda, A. et al., Vaccine 1991; 9:741-45) and acellular vaccines induce pertactin cell mediated immunity (Podda, A. et al., Vaccine 1991; 9:741-45). Pertactin protects mice against aerosol challenge with B. pertussis (Roberts, M. et al., Vaccine 1992:10:43-48) and in combination with FHA, protects in the intracerebral challenge test against B. pertussis (Novotny, P. et at; J. Infect. Dis. 1991; 164:114-22). Passive transfer of polyclonal or monoclonal anti-pertactin antibodies also protects mice against aerosol challenge (Shaun. R. D. et al., J. Exp. Med. 1990; 171:63-73).

Filamentous Hemagglutinin

Filamentous haemagglutinin (FHA) is a large (220 kDa) non-toxic polypeptide which mediates attachment of B. pertussis to ciliated cells of the upper respiratory tract during bacterial colonization (Tuomanen, E. and Weiss, A., J. Infect. Dis., 1985; 152:118-25). Vaccination with whole-cell or acellular pertussis vaccines generates anti-FHA antibodies and acellular vaccines containing FHA also induce a cell mediated immune response to FHA (Gearing, A. et al., FEMS Microbial. Immunol. 1989; 47:205-12; Thomas, M. G. et al., J. Infect. Dis. 1989; 160:838-45; Di Tommaso, A. et al., Infect. Immun. 1991; 59:3313-15; Tomoda.; T. et al.; J. Infect. Dis. 1992; 166:908-10).

Fimbriae Types 2 and 3

Serotypes of B. pertussis are defined by their agglutinating fimbriae. The WHO recommends that whole-cell vaccines include types 1, 2 and 3 agglutinogens (Aggs) since they are not cross-protective (Robinson, A. et at, Vaccine 1985; 3:11-22). Agg 1 is non-fimbrial and is found on all B. pertussis strains while the serotype 2 and 3 Aggs are fimbrial. Natural infection or immunization with whole-cell or acellular vaccines induces anti-Agg antibodies (Thomas, M. G. et al., J. Infect. Dis. 1989; 160:838-45; Edwards, K. M. et al., Pediatr. Res, 1992; 31:91A). A specific cell-mediated immune response can be generated in mice by Agg 2 and Agg 3 after aerosol infection (Petersen, J. W. et al., immun. 1992; 60:4563-70). Aggs 2 and 3 are protective in mice against respiratory challenge and human colostrum containing anti-agglutinogens will also protect in this assay (Oda, M. et al., Infect. Immun. 1985; 47:441-45; Robinson, A. et al, Develop. Biol. Stand. 1985; 61:165-72, Robinson; A. et al Vaccine 1989; 7:321-24).

TLR Agonist

The aP booster vaccines described herein include a toll-like receptor (TLR) agonist. The TLR agonist is a compound that can agonize or activate TLRs. TLRs are an important component of the host's pathogen sensing mechanism (Janeway et al., Annu. Rev. Immunol. 2002; 20:197-216); Akira et al., Nat Rev Immunol. 2004; 4:499-511). TLRs are typically classified into two families based on their localization: TLRs 1, 2, and 4-6 are expressed on the cell surface and sense bacterial cell wall components whereas TLRs 3 and 7-9 are expressed in endosomes and sense viral or bacterial nucleic acids (Kawasaki et al., Front Immunol. 2014:5:461). The molecular structures recognized by TLRs have been evolutionarily conserved and are expressed by a wide variety of infectious microorganisms (Janeway et al., Annu. Rev. Immunol. 2002; 20:197-216); Akira et al., Nat Rev Immunol. 2004; 4:499-511). The innate immune response elicited by TLR activation is characterized by the production of pro-inflammatory cytokines, chemokines, type I interferons and anti-microbial peptides. This innate response promotes and modulates the adaptive immune system. A common result is the expansion of antigen-specific B cells that produce high affinity antibodies and of cytotoxic T cells including long-lasting memory cells that protect against subsequent infection through enhanced cytotoxic function targeting the effector phase (Wille-Reece et al., J Exp Med. 2006; 203:1249-58); Xiao et al., J Immunol. 2013; 190:5866-73). TLR signaling appears to play an important role in many aspects of the innate immune response.

By including the TLR agonist, the aP booster vaccine is surprisingly able to shift a Th2 biased immune response, established by a previously administered aP vaccine, to a Th1-biased immune response. Preferably, the TLR agonist is an agonist of a human TLR.

In certain embodiments, the TLR agonist is a TLR4 agonist and preferably an agonist of human TLR4. In one embodiment, the TLR4 agonist is E6020, a synthetic phospholipid dimer that mimics the physicochemical and biological properties of the natural lipid A derived from Gram-negative bacteria (Ishizaka et al., Expert Rev Vaccines. 2007; (5):773-84). E6020 is dodecanoic acid, (1R,6R, 22R, 27R)-1,27-dihexyl-9,19-dihydroxy-9,19-dioxido-14-oxo-6,22-bis [(1,3-di oxotetradecyl)amino]-4,8,10, 18, 20, 24-hexaoxa-13,15-di aza-9,19-dephosphaheptacosane-1,27-diyl ester, disodium salt (C₈₃H₁₅₈N₄O₁₉P₂Na₂). The chemical synthesis of E6020 is a reproducible and well-controlled manufacturing process yielding a highly pure chemical compound. E6020 has the following chemical structure:

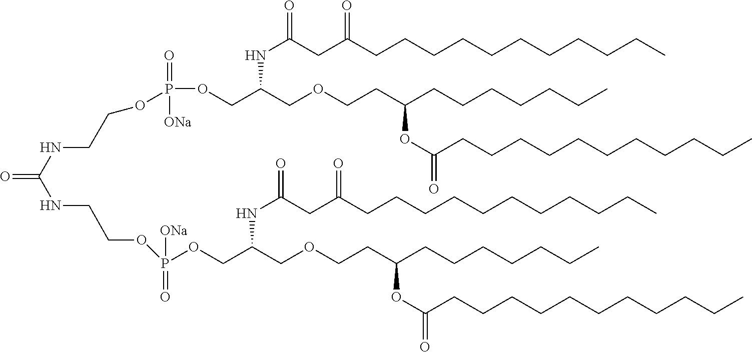

E6020 interacts with TLR4 and has been evaluated as an adjuvant in preclinical studies, combined with emulsions, liposomes or aluminum salts. E6020 has been reported to enhance IgG2a, which in mice is associated with Th1 activation. E6020 has also been shown to enhance granulocyte-macrophage colony-stimulating factor (GM-CSF), IL-1, IL-6 and TNF-α in human peripheral blood mononuclear cells (PBMCs) and mouse spleen (Ishizaka et al., Expert Rev Vaccines. 2007; (5):773-84).

In other embodiments the TLR agonist is a TLR9 agonist and preferably an agonist of human TLR9. For example, the TLR9 agonist may be a CpG oligodeoxynucleotide (“ODN”). As used herein, a “CpG oligonucleotide” or “CpG ODN” is a single stranded DNA molecule that contains at least one central unmethylated CG dinucleotide embedded within specific flanking regions. CpG ODNs are present at high frequency in bacterial DNA and possess an immunostimulatory effect.

In humans, CpG ODNs have been categorized into 4 distinct classes based on differences in structure and the nature of the immune response they induce. Although each class contains at least one central unmethylated CG dinucleotide plus flanking regions, they differ in structure and immunological activity. Class B ODNs (also referred to as “K” type) contain from one to five CpG motifs typically on a phosphorothioate backbone. Phosphorothioate is a non-naturally occurring internucleoside linking group that replaces the phosphodiester linkage found in naturally occurring DNA and enhances resistance to nuclease digestion and substantially prolongs in vivo half-life. Class B ODNs trigger plasmacytoid dendritic cells to differentiate and produce TNFα and stimulate B cells to proliferate and secrete IgM.

Class A ODNs (also referred to as “D” type) have a phosphodiester core flanked by phosphorothioate terminal nucleotides. They include a single CpG motif flanked by palindromic sequences that are able to form stem-loop structures. Class A ODN also have poly G motifs at the 3′ and 5′ ends that promote concatamer formation. Class A ODNs trigger plasmacytoid dendritic cells to mature and secrete IFNα but have no effect on B cells. Class C ODNs resemble Class B in that they are composed entirely of phosphorothioate nucleotides but resemble Class A in containing palindromic CpG motifs that can form stem loop structures or dimers. Class C ODNs stimulate B cells to secrete IL-6 and plasmacytoid dendritic cells to produce IFNα. Class P CpG ODNs are highly ordered structures containing double palindromes that can form hairpins at their GC-rich 3′ ends as well as concatamerize due to the presence of the 5′ palindromes.

In certain embodiments, the CpG ODN used in the aP booster vaccines is a Class B CpG ODN. In certain embodiments, the CpG ODN used in the aP booster vaccines is a Class A CpG ODN. In certain embodiments, the CpG ODN used in the aP booster vaccines is a Class C CpG ODN. In certain embodiments, the CpG ODN used in the aP booster vaccines is a Class P CpG ODN.

In certain embodiments, the CpG ODN contains at least one phosphorothioate linkage. In certain embodiments, all the nucleotides in the CpG ODN are linked with a phosphorothioate linkage. In certain embodiments, the CpG ODN contains 1-5 CG dinucleotides. In certain embodiments, the CpG ODN contains 1 CG dinucleotide. In certain embodiments, the CpG ODN contains 2 CG dinucleotides. In certain embodiments, the CpG ODN contains 3 CG dinucleotides. In certain embodiments, the CpG ODN contains 4 CG dinucleotides. In certain embodiments, the CpG ODN contains 5 CG dinucleotides. In certain embodiments, the CpG ODN is 18-28 nucleotides in length.

In one embodiment, the CpG ODN is ISS1018 (Higgins et al., Exp Rev Vaccines, 2007; 6(5):747-59), a 22-mer oligonucleotide having the following nucleotide sequence: 5′-TGACTGTGAACGTTCGAGATGA-3′ (SEQ ID NO: 1). All of the nucleotide bases in ISS1018 are linked with phosphorothioate linkages. As used herein, “CpG1018” is used interchangeably with ISS1018.

Aluminum Salt

Adjuvants, such as aluminum salts, have been used to enhance the immune response to various antigens. Aluminum salts that can be used as adjuvants include, but are not limited to, aluminum hydroxide/oxyhydroxide (A100H), aluminum phosphate (AlPO₄), aluminum hydroxyphosphate sulfate (AAHS) and/or potassium aluminum sulfate. These aluminum salts have a long history of use in vaccines.

As discussed elsewhere in this application, one or more of the tetanus toxoid, the diphtheria toxoid, and the acellular B. pertussis antigens of the aP booster vaccine can be adsorbed to the aluminum salt. In certain embodiments, all of the vaccine antigens in the aP booster vaccine are adsorbed to the aluminum salt. For example, in one embodiment, the tetanus toxoid, the diphtheria toxoid, PT, FHA, PT, and FIM2,3 are adsorbed to A100H. In another embodiment, the tetanus toxoid, the diphtheria toxoid, PT, FHA, PT, and FIM2,3 are adsorbed to AlPO₄. In yet another embodiment, one or more of the vaccine antigens in the aP booster vaccine are adsorbed to AlOOH and one or more of the vaccine antigens in the aP booster vaccine are adsorbed to AlPO₄.

In certain embodiments, the TLR agonist is formulated with the aluminum salt. Typically, the TLR4 agonist, such as E6020, is formulated with A100H. In other embodiments, the TLR4 agonist, such as E6020, is formulated with AlPO₄. Typically, the TLR9 agonist, such as the CpG ODN (e.g., CpG1018), is formulated with A100H. In other embodiments, the TLR9 agonist, such as the CpG ODN (e.g., CpG1018), is formulated with AlPO₄.

In certain embodiments, the aP booster vaccine comprises a tetanus toxoid, a diphtheria toxoid, gdPT, FHA, PT, FIM2,3, and a TLR4 agonist (e.g., E6020) and each of the tetanus toxoid, the diphtheria toxoid, gdPT, FHA, PT, FIM2,3, is adsorbed to A100H, and the TLR4 agonist (e.g., E6020) is formulated with A100H. In certain embodiments, the aP booster vaccine comprises a tetanus toxoid, a diphtheria toxoid, gdPT, FHA, PT, FIM2,3, and a TLR9 agonist (e.g., CpG ODN, such as CpG1018) and each of the tetanus toxoid, the diphtheria toxoid, gdPT, FHA, PT, FIM2,3, is adsorbed to A100H, and the TLR9 agonist (e.g., CpG ODN, such as CpG1018) is formulated with A100H.

In certain embodiments, the aP booster vaccine includes both AlOOH and AlPO₄ and antigens in the vaccine may be adsorbed to one or both of these aluminum salts.

Methods for adsorbing diphtheria toxoid, tetanus toxoid, and pertussis antigens to aluminum salt, such as AlOOH and AlPO₄ are known in the art.

Other Antigens

In addition to the tetanus toxoid, the diphtheria toxoid, and the acellular B. pertussis antigens, the aP booster vaccine can contain one or more additional antigens, including, but not limited to a Haemophilus influenzae type-b saccharide (Hib) conjugate, a hepatitis B virus surface antigen (HBsAg) and/or an inactivated polio virus (IPV).

Haemophilus influenzae type-b causes bacterial meningitis. Hib vaccines are typically formulated using Hib conjugated to a carrier protein to enhance its immunogenicity, especially in children. Typically, the carrier protein is tetanus toxoid, diphtheria toxoid, H influenza protein D, or an outer membrane protein complex from serogroup B. meningococcus. But any appropriate carrier protein can be used. Methods of making Hib conjugates are known in the art. For example, PENTACEL® contains H. influenzae type b capsular polysaccharide (polyribosyl-ribitol-278 phosphate [PRP]) covalently bound to tetanus toxoid. The Hib conjugate is typically adsorbed to an aluminum salt (e.g., AlOOH or AlPO₄).

Hepatitis B virus (HBV) causes viral hepatitis, a potentially life-threatening liver infection. The infectious HBV virion has a spherical, double-shelled structure, consisting of a lipid envelope containing HBsAg that surrounds an inner nucleocapsid composed of hepatitis B core antigen (HBcAg) complexed with virally encoded polymerase and the viral DNA genome. HBsAg is a polypeptide that typically has a length of 226 amino acids and a molecular weight of about 24 kDa. HBV vaccines typically contain HBsAg. Thus, methods of making HBsAg and vaccines comprising HBsAg are well known in the art. The HBsAg is typically adsorbed to an aluminum salt (e.g., AlOOH or AlPO₄)

Poliomyelitis is a disease caused by any one of three types of polio virus: poliovirus Type 1 (e.g., Mahoney strain), poliovirus Type 2 (e.g., MEF-1 strain), and poliovirus Type 3 (e.g., Saukett strain). Polioviruses can be grown in cell culture using known techniques, followed by purification of virions using techniques, such as ultrafiltration, diafiltration, and chromatography. Next, the virions are inactivated using, for example, formaldehyde. Typically, each type of poliovirus is grown individually, purified from the cell culture, and inactivated before combining them to produce a trivalent poliovirus composition. Typically, the inactivated poliovirus is not adsorbed onto an aluminum salt prior to formulating the vaccine. However, the inactivated poliovirus may become adsorbed onto any aluminum salt in the vaccine that is not adsorbed to another vaccine antigen.

Acellular Pertussis Booster Vaccine

The aP booster vaccine is an immunogenic composition that includes one or more antigens but not all antigens which are derived from or homologous to, antigens from B. pertussis and other pathogens (e.g., Corynebacterium diphtheria, Clostridium tetani, etc.). Such a vaccine is substantially free of intact pathogenic particles or the lysate of such particles. Thus, the aP booster vaccine can be prepared from at least partially purified, or substantially purified, immunogenic polypeptides from a pathogen of interest or their analogs. Methods of obtaining an antigen or antigens in the vaccine include standard purification techniques, recombinant production, or chemical synthesis.

In various embodiments, the one or more antigens are formulated into a unit dose of an aP booster vaccine. A “unit dose” as used herein refers to an amount of vaccine that is administered to a subject in a single administration. Typically, this amount is present in a volume of 0.1-2 milliliters, e.g., 0.2-1 milliliters, and typically 0.5 milliliters. The indicated amounts may, thus, for instance, be present at a concentration of micrograms per 0.5 milliliters bulk vaccine. In certain embodiments a (single) unit dose thus equals 0.5 milliliters.

As described herein, the aP booster vaccine comprises a tetanus toxoid, a diphtheria toxoid, and the following acellular B. pertussis antigens: detoxified pertussis toxin, filamentous hemagglutinin, pertactin, and fimbriae Types 2 and 3. The aP booster vaccine also contains a TLR agonist, such as a TLR4 (e.g., E6020) or a TLR9 agonist (e.g., CpG1018) and an aluminum salt, such as AlOOH or AlPO₄.

The acellular pertussis antigens are typically prepared by isolation from B. pertussis cultures grown in liquid culture medium. Any liquid culture medium known in the art for cultivating Bordetella cells may be used. In various embodiments, a complex medium is used. As used herein, a “complex medium” refers to a medium that contains peptone digests or extracts of plant or animal-origin. Examples of complex media suitable for use with the present methods include e.g., Hornibrook's medium, Cohen-Wheeler medium, B2 Medium, or other similar liquid culture media. A modified Stainer & Scholte medium that also includes dimethyl beta-cyclodextrin and casamino acids is another example suitable for use. Stainer et al., J Gen Microbiol, 1970, 63:211-20. Pertussis toxin, filamentous hemagglutinin, and pertactin are typically isolated separately from the supernatant culture medium. Fimbriae types 2 and 3 are typically extracted and co-purified from the bacterial cells. The pertussis antigens can be purified from the supernatant and/or bacterial cells using any conventional methods, including, for example, sequential filtration, salt-precipitation, ultrafiltration and chromatography.

The tetanus toxoid (TT) is typically present in the aP booster vaccine in an amount from about 8-12 limit of flocculation (Lf)/mL. In certain embodiments, the TT is present in an amount of 9-11 Lf/mL. In certain embodiments, the TT is present in an amount of 10 Lf/mL. As measured per unit dose form, where a unit dose is 0.5 mL, the TT is typically present in an amount of about 4-6 Lf. In certain embodiments of the 0.5 mL dose form, the TT is present in an amount of 4.5-5.5 Lf. In certain embodiments of the 0.5 mL dose form, the TT is present in an amount of 5 Lf. In the aP booster vaccine, TT is typically adsorbed to an aluminum salt. Typically, TT is adsorbed onto A100H. In other embodiments, TT may be adsorbed onto AlPO₄.

The diphtheria toxoid (DT) is typically present in the aP booster vaccine in an amount from about 3-8 Lf/mL. In certain embodiments, the DT is present in an amount of 3-6 Lf/mL. In certain embodiments, the DT is present in an amount of 4-5 Lf/mL. In certain embodiments, the DT is present in an amount of 4 Lf/mL. As measured per unit dose form, where a unit dose is 0.5 mL, the DT is typically present in an amount of about 1.5-4 Lf. In certain embodiments of the 0.5 mL dose form, the DT is present in an amount of 1.5-3 Lf. In certain embodiments of the 0.5 mL dose form, the DT is present in an amount of 2-2.5 Lf. In certain embodiments of the 0.5 mL dose form, the DT is present in an amount of 2 Lf. In the aP booster vaccine, DT is typically adsorbed to an aluminum salt. Typically, DT is adsorbed onto A100H. In other embodiments, DT may be adsorbed onto AlPO₄.

The detoxified pertussis toxin (PT) is typically present in the aP booster vaccine in an amount from about 4-30 μg/mL. In certain embodiments, PT is chemically detoxified PT and is present in an amount of 4-10 μg/mL. In certain embodiments, the PT is genetically-detoxified PT (gdPT) and is present in an amount of about 16-24 μg/mL. In certain embodiments, the gdPT and is present in an amount of 18-22 μg/mL. In certain embodiments, the gdPT is present in an amount of 20 μg/mL. As measured per unit dose form, where a unit dose is 0.5 mL, the PT is typically present in an amount of about 2-15 μg. In certain embodiments of the 0.5 mL dose form, the PT is present in an amount of 2-5 μg. In certain embodiments of the 0.5 mL dose form, the PT is gdPT and is present in an amount of 8-12 μg. In certain embodiments of the 0.5 mL dose form, the gdPT is present in an amount of 9-11 μg. In certain embodiments of the 0.5 mL dose form, the gdPT is present in an amount of 10 μg. In other embodiments, the PT is present in an amount ranging from 2-50 μg, 5-40 μg, 10-30 μg, or 20-25 μg per unit dose. In the aP booster vaccine, PT is typically adsorbed to an aluminum salt. In certain embodiments, PT is adsorbed onto A100H. In certain embodiments, PT is adsorbed onto AlPO₄.

The filamentous hemagglutinin (FHA) is typically present in the aP booster vaccine in an amount from about 5-15 μg/mL. In certain embodiments, the FHA is present in an amount of 8-12 μg/mL. In certain embodiments, the FHA is present in an amount of 10 μg/mL. As measured per unit dose form, where a unit dose is 0.5 mL, the FHA is typically present in an amount of about 2.5 to 7.5 μg. In certain embodiments of the 0.5 mL dose form, the FHA is present in an amount of 4-6 μg. In certain embodiments of the 0.5 mL dose form, the FHA is present in an amount of 5 μg. In other embodiments, the FHA is present in an amount ranging from 2-50 μg, 5-40 μg, 10-30 μg, or 20-25 μg per unit dose. In the aP booster vaccine, FHA is typically adsorbed to an aluminum salt. In certain embodiments, FHA is adsorbed onto A100H. In certain embodiments, FHA is adsorbed onto AlPO₄.

The pertactin (PRN) is typically present in the aP booster vaccine in an amount from about 5-15 μg/mL. In certain embodiments, the PRN is present in an amount of 8-12 μg/mL. In certain embodiments, the PRN is present in an amount of 10 μg/mL. As measured per unit dose form, where a unit dose is 0.5 mL, the PRN is typically present in an amount of about 2.5 to 7.5 μg. In certain embodiments of the 0.5 mL dose form, the PRN is present in an amount of 4-6 μg. In certain embodiments of the 0.5 mL dose form, the PRN is present in an amount of 5 μg. In other embodiments, the PRN is present in an amount ranging from 0.5-100 μg, 1-50 μg, 2-20 μg, 3-30 μg, or 5-20 μg per unit dose. In the aP booster vaccine, PRN is typically adsorbed to an aluminum salt. In certain embodiments, PRN is adsorbed onto A100H. In certain embodiments, PRN is adsorbed onto AlPO₄.

The fimbriae types 2 and 3 (FIM2,3) are typically present in the aP booster vaccine in an amount from about 10-20 μg/mL. In certain embodiments, the FIM2,3 is present in an amount of 14-16 μg/mL. In certain embodiments, the FIM2,3 is present in an amount of 15 μg/mL. In certain embodiments, the weight ratio of FIM 2 to FIM 3 is from about 1:3 to about 3:1, e.g., from about 1:1 to about 3:1, e.g., from about 1.5:1 to about 2:1. As measured per unit dose form, where a unit dose is 0.5 mL, the FIM2,3 is typically present in an amount of about 5-10 μg. In certain embodiments of the 0.5 mL dose form, the FIM2,3 is present in an amount of 7-8 μg. In certain embodiments of the 0.5 mL dose form, the FIM2,3 is present in an amount of 7.5 μg. In other embodiments, FIM2/3 is present in an amount ranging from 1-100 μg per unit dose, such as 3-50 μg, or 3-30 μg per unit dose. In the aP booster vaccine, FIM2,3 are typically adsorbed to an aluminum salt. In certain embodiments, FIM2,3 is adsorbed onto A100H. In certain embodiments, FIM2,3 is adsorbed onto AlPO₄.

Typically, the aP booster vaccine includes an aluminum salt, such as AlOOH or AlPO₄, which is used to adsorb one or more of the vaccine antigens and/or to formulate the TLR agonist. In certain embodiments, the aluminum salt is present in an amount from about 0.25-0.75 mg/mL, 0.25-0.35 mg/mL, or 0.6-0.7 mg/mL. In certain embodiments, the aluminum salt is present in an amount of 0.66 mg/mL. As measured per unit dose form, where a unit dose is 0.5 mL, the aluminum salt is typically present in an amount of 0.125-0.375 mg, 0.125-0.175 mg, or 0.3-0.35 mg. In certain embodiments, the 0.5 mL unit dose form contains 0.33 mg of aluminum salt. In certain embodiments, the aluminum salt is A100H. In other embodiments, the aluminum salt is AlPO₄.

When the TLR4 agonist is E6020, it can be present in an amount of no more than 10 μg/ml. In certain embodiments, E6020 is present in an amount of 0.5-5 μg/ml. In certain embodiments, E6020 is present in an amount of no more than 2 μg/ml. As measured per unit dose form, where a unit dose is 0.5 mL, E6020 is typically present in an amount of no more than 5 μg. In certain embodiments of the 0.5 mL dose form, E6020 is present in an amount of about 0.25-2.5 μg. In certain embodiments of the 0.5 mL dose form, E6020 is present in an amount of no more than 1 μg.

In certain embodiments, E6020 is formulated with an aluminum salt. Typically, the aluminum salt is A100H. In other embodiments, the aluminum salt may be AlPO₄.

When the TLR9 agonist is CpG1018, it can be present in an amount of about 250-750 μg/ml. In certain embodiments, CpG1018 is present in an amount of 400-600 μg/ml. In certain embodiments, CpG1018 is present in an amount of 500 μg/ml. As measured per unit dose form, where a unit dose is 0.5 mL, CpG1018 is typically present in an amount of about 125-375 μg. As measured per unit dose form, where a unit dose is 0.5 mL, CpG1018 is present in an amount of 200-300 μg. In certain embodiments of the 0.5 mL dose form, CpG1018 is present in an amount of 250 μg.

In certain embodiments, CpG1018 is formulated with an aluminum salt. Typically, the aluminum salt is A100H. In other embodiments, the aluminum salt may be AlPO₄.

In certain embodiments, the aP booster vaccine comprises TT in an amount of 8-12 Lf/mL, DT in an amount of 3-8 Lf/mL, gdPT in an amount of 16-24 μg/mL, FHA in an amount of 5-15 μg/mL, PRN in an amount of 5-15 μg/mL, FIM2,3 in an amount from about 10-20 μg/mL, AlOOH in an amount of 0.25-0.75 mg/mL, and a TLR4 agonist, such as E6020, in an amount of no more than 10 μg/ml.

In certain embodiments, the aP booster vaccine comprises TT in an amount of 8-12 Lf/mL, DT in an amount of 3-8 Lf/mL, gdPT in an amount of 16-24 μg/mL, FHA in an amount of 5-15 μg/mL, PRN in an amount of 5-15 μg/mL, FIM2,3 in an amount from about 10-20 μg/mL, AlOOH in an amount of 0.25-0.75 mg/mL, and a TLR9 agonist, such as CpG1018, in an amount of 250-750 μg/ml.

In certain embodiments, the aP booster vaccine comprises TT in an amount of 9-11 Lf/mL, DT in an amount of 4-6 Lf/mL, gdPT in an amount of 18-22 μg/mL, FHA in an amount of 8-12 μg/mL, PRN in an amount of 8-12 μg/mL, FIM2,3 in an amount of 14-16 μg/mL, AlOOH in an amount of 0.6-0.7 mg/mL, and a TLR4 agonist, such as E6020, in an amount of 0.5-5 μg/ml.

In certain embodiments, the aP booster vaccine comprises TT in an amount of 9-11 Lf/mL, DT in an amount of 4-6 Lf/mL, gdPT in an amount of 18-22 μg/mL, FHA in an amount of 8-12 μg/mL, PRN in an amount of 8-12 μg/mL, FIM2,3 in an amount of 14-16 μg/mL, AlOOH in an amount of 0.6-0.7 mg/mL, and a TLR9 agonist, such as CpG1018, in an amount of 400-600 μg/ml.

In certain embodiments, the aP booster vaccine comprises the following components at the indicated concentrations, as set forth in Table 1:

TABLE 1

| Component | Amount | |
|---|---|---|
| Tetanus toxoid | 10 | Lf/mL |
| Diphtheria toxoid | 4-5 | Lf/mL |
| Genetically-detoxified Pertussis Toxin | 20 | μg/mL |
| Filamentous Hemagglutinin | 10 | μg/mL |
| Pertactin | 10 | μg/mL |
| Fimbriae Types 2 and 3 | 15 | μg/mL |
| Aluminum salt (AlOOH) | 0.66 | mg/mL Al |

| | |
|---|---|
| TLR Agonist | E6020 (0.5-5 μg/mL) or |
| | CpG1018 (0.5 mg/mL) |

In certain embodiments of the aP booster vaccine set forth in Table 1, the diphtheria toxoid is present in an amount of 4 Lf/mL. In certain embodiments of the aP booster vaccine set forth in Table 1, the diphtheria toxoid is present in an amount of 5 Lf/mL.

In certain embodiments, the aP booster vaccine is in a unit dose form for administration to a human subject and comprises the following components per 0.5 mL dose: TT in an amount of 4-6 Lf, DT in an amount of 1.5-4 Lf, gdPT in an amount of 8-12 μg, FHA in an amount of 2.5-7.5 PRN in an amount of 2.5-7.5 μg, FIM2,3 in an amount from about 5-10 μg, AlOOH in an amount of 0.125-0.375 mg, and a TLR4 agonist, such as E6020, in an amount of no more than 5 μg.

In certain embodiments, the aP booster vaccine is in a unit dose form for administration to a human subject and comprises the following components per 0.5 mL dose: TT in an amount of 4-6 Lf, DT in an amount of 1.5-4 Lf, gdPT in an amount of 8-12 μg, FHA in an amount of 2.5-7.5 PRN in an amount of 2.5-7.5 μg, FIM2,3 in an amount from about 5-10 μg, AlOOH in an amount of 0.125-0.375 mg, and a TLR9 agonist, such as CpG1018, in an amount of 125-375 μg.

In certain embodiments, the aP booster vaccine is in a unit dose form for administration to a human subject and comprises the following components per 0.5 mL dose: TT in an amount of 4.5-5.5 Lf, DT in an amount of 1.5-3 Lf, gdPT in an amount of 9-11 μg, FHA in an amount of 4-6 μg, PRN in an amount of 4-6 μg, FIM2,3 in an amount from about 7-8 μg, AlOOH in an amount of 0.3-0.35 mg, and a TLR4 agonist, such as E6020, in an amount of 0.25-2.5 μg.

In certain embodiments, the aP booster vaccine is in a unit dose form for administration to a human subject and comprises the following components per 0.5 mL dose: TT in an amount of 4.5-5.5 Lf, DT in an amount of 1.5-3 Lf, gdPT in an amount of 9-11 μg, FHA in an amount of 4-6 μg, PRN in an amount of 4-6 μg, FIM2,3 in an amount from about 7-8 μg, AlOOH in an amount of 0.3-0.35 mg, and a TLR9 agonist, such as CpG1018, in an amount of 200-300 μg.

In certain embodiments, the aP booster vaccine is in a unit dose form for administration to a human subject and comprises the following components per 0.5 mL dose, as set forth in Table 2:

TABLE 2

| Component | Amount | |
|---|---|---|
| Tetanus toxoid | 5 | Lf |
| Diphtheria toxoid | 2-2.5 | Lf |
| Genetically-detoxified Pertussis Toxin | 10 | μg |
| Filamentous Hemagglutinin | 5 | μg |
| Pertactin | 5 | μg |
| Fimbriae Types 2 and 3 | 7.5 | μg |
| Aluminum salt (AlOOH) | 0.33 | mg Al |

| | |
|---|---|
| TLR Agonist | E6020 (0.25-2.5 μg) or |
| | CpG1018 (250 μg) |

In certain embodiments of the aP booster vaccine set forth in Table 1, the diphtheria toxoid is present in an amount of 2 Lf. In certain embodiments of the aP booster vaccine set forth in Table 1, the diphtheria toxoid is present in an amount of 2.5 Lf.

In certain embodiments, the aP booster vaccine is formulated to contain antigens other than tetanus toxoid, diphtheria toxoid, or Bordetella antigens. For example, in certain embodiments, the aP booster vaccine comprises one or more of the following: Haemophilus influenzae type-b oligosaccharide or polysaccharide (Hib) conjugate, a hepatitis B virus surface antigen (HBsAg) and/or an inactivated polio virus (IPV).

The aP booster vaccine described herein may be formulated as an injectable, liquid solution or emulsion. For example, the tetanus toxoid, diphtheria toxoid, and Bordetella antigens may be mixed with pharmaceutically acceptable excipients which are compatible with the antigens. Such excipients may include water, saline, dextrose, glycerol, ethanol, and combinations thereof. The aP booster vaccine may further contain auxiliary substances, such as wetting or emulsifying agents, pH buffering agents, or adjuvants to enhance the effectiveness thereof.

Typically, the aP booster vaccine will be formulated in aqueous form. Typically, the components of the aP booster vaccine will be diluted with Tris-buffered saline to give the desired final concentrations. Alternatively, the diluent for formulation can be water for injection.

Methods of Administering aP Booster Vaccine

The aP booster vaccines described herein are suitable for administration to a human subject. Thus, one aspect is directed to a method of inducing an immune response in a human subject, the method comprising administering to the human subject an aP booster vaccine as described herein. Also described is the aP booster vaccine for use in inducing an immune response in a human subject and/or for reorienting a Th2-biased immune response towards a Th-1 biased immune response or a Th1/Th17-biased immune response in a human subject.

Typically, the human subject has received either a wP vaccine, no pertussis vaccine, or an aP priming vaccine prior to administering the aP booster vaccine, which aP priming vaccine induces a Th2-biased immune response. Typically, when a human subject who has received an aP priming vaccine receives an aP booster vaccine that does not contain a TLR agonist (e.g., ADACEL®), the non-TLR agonist containing aP booster vaccine boosts the Th2-biased immune response induced by the aP priming vaccine, maintaining the Th2 bias induced by aP priming vaccine. By contrast, the modified, TLR agonist containing aP booster vaccine described herein unexpectedly reorients or shifts the Th2-biased immune response induced by the aP priming vaccine towards a Th1-biased immune response or Th1/Th17-bi as ed immune response.

In certain embodiments, the Th1-biased immune response is characterized by one or more of decreased IL-5 production, increased IFN-γ production, or a lower IgG1/IgG2a ratio, as compared to the immune response induced by the aP priming vaccine or an aP booster vaccine that does not contain a TLR agonist (e.g., ADACEL®). In certain embodiments, the Th1-biased immune response is characterized by decreased IL-5 production and/or a lower IgG1/IgG2a ratio, as compared to the immune response induced by the aP priming vaccine or an aP booster vaccine that does not contain a TLR agonist (e.g., ADACEL®).

In certain embodiments, the Th1/Th17 biased immune response is characterized by increased IL-17 production and one or more of decreased IL-5 production and/or a lower IgG1/IgG2a ratio as compared to the immune response induced by the aP priming vaccine or an aP booster vaccine that does not contain a TLR agonist (e.g., ADACEL®).

The aP priming vaccine may be administered as a single dose or a series of multiple doses (e.g., 2, 3, 4, or 5 doses) prior to administering the aP booster vaccine. Typically, the aP priming vaccine is administered as a series of doses, especially for children. For example, in certain embodiments, the aP priming vaccine is administered as a series of five doses in infants and children between 6 weeks and 6 years of age. The aP priming vaccine can also be administered as a series of four doses in infants and children between 6 weeks and 4 years of age. Typically, the primary immunization schedule for a child includes administering an aP priming vaccine at 2 months, 4 months, 6 months, 15-20 months, and 4-6 years of age.

The aP booster vaccine is typically administered after the primary immunization schedule is complete. In certain embodiments, the aP booster vaccine is administered to a human subject who is 10 years of age or older. In certain embodiments, the aP booster vaccine is administered to a human subject who is 4 years of age or older.

Typically, the aP vaccine booster is administered by intramuscular injection.

In certain embodiments, the aP priming vaccine comprises a tetanus toxoid, a diphtheria toxoid, a detoxified pertussis toxin, filamentous hemagglutinin, pertactin, and optionally FIM2,3 with the proviso that the aP priming vaccine does not contain a TLR agonist.

In certain embodiments, the aP priming vaccine includes one or more doses of DAPTACEL®, PENTACEL®, QUADRACEL®, INFANRIX INFANRIX-HEXA®, KINRIX®, PEDIARIX®, or VAXELIS®. In certain embodiments, the aP priming vaccine comprises DAPTACEL®. In certain embodiments, the aP priming vaccine comprises PENTACEL® or QUADRACEL®. In certain embodiments, the aP priming vaccine comprises INFANRIX® or INFANRIX-HEXA®. In certain embodiments, the aP priming vaccine comprises KINRIX® or PEDIARIX®. In certain embodiments, the aP priming vaccine comprises VAXELIS®.

### EXAMPLES

Materials and Methods—General

B. pertussis Challenge

Bordetella pertussis 18323 were grown on Bordet-Gengou agar (Difco) supplemented with 1% glycerol, 20% defibrinated sheep blood (Sanofi Pasteur, Alba La Romaine). After 24 h at 36° C., colonies were transferred into 1% Casamino Acid (Difco) buffer and the optical density of the bacterial suspension was measured. 5×10⁶ colony-forming units (CFU) were instilled intranasally in a volume of 30 μl into mice anesthetized by intramuscular injection of Imalgen (ketamine 60 mg/kg; Merial SAS) and Rompun (Xylaxine 4 mg/kg; Bayer). Mice were then euthanized by intraperitoneal injection of Dolethal (pentobarbital 180 mg/kg; Vetoquinol SA) 2 hours after infection for quantification of the initial numbers of viable B. pertussis CFUs in the lungs and at either days 3, 7, 14 and 21 or days 1, 2, 3, 7 and 14 for determination of bacterial colonization. Briefly, lungs homogenates were plated onto Bordet-Gengou agar plates and the number of CFUs was counted after 4 days of incubation at 36° C. The measure of protective efficacy was expressed as a ratio of the area under the clearance curve (AUC) between naive control and immunized mice.

Adjuvant Formulations (all Examples)

The modified Tdap (gdPT+CpG1018-A100H) booster and the modified Tdap (gdPT+E6020-A100H) booster (for mice) contained 10 Lf/ml TT, 4 Lf/ml DT, 20 μg/ml gdPT, 10 μg/ml PRN, 15 μg/ml FIM2/3, 10 μg/ml FHA and 0.66 mg/ml aluminum (A100H) with either 500 μg/ml CpG1018 (TLR9 agonist) or 10 μg/ml E6020 (TLR4 agonist).

### Antigens, Adjuvants and Immunizations (all Examples)

Mice were primed intra-muscularly with ⅕th of a human dose (50 μl in both hind legs) of the pediatric diphtheria-tetanus-acellular pertussis (aP) DAPTACEL® vaccine (Sanofi Pasteur), containing 10 μg chemically detoxified PT, 5 μg FHA, 3 μg PRN and 5 μg FIM2,3 in addition to tetanus and diphtheria toxoids (referred to in the examples and figures as DTaP for short), or of the diphtheria-tetanus-whole bacterial-cell pertussis (wP) D.T.COQ/D.T.P vaccine (Sanofi Pasteur) containing ≥4 I.U. B. pertussis inactivated by heat in presence of thiomersal (referred to in the examples and figures as DTwP for short). Recipient mice were boosted intra-muscularly with ⅕th of a human dose (50 μl in both hind legs) of the following formulations: pediatric diphtheria-tetanus-aP ADACEL® vaccine (Sanofi Pasteur) containing 2.5 μg chemically detoxified PT, 5 μg FHA, 3 μg PRN and 5 μg FIM2,3 in addition to tetanus and diphtheria toxoids (referred to in the examples and figures as Tdap for short); D.T.COQ/D.T.P vaccine (i.e., DTwP); or the modified Tdap booster formulations described above (referred to in the examples and figures as modified Tdap or mTdap for short).

Fluorospot (All):

Splenic IFN-γ, IL-5 or IL-17 cytokine secreting cells were detected using a FluoroSpot assay (ELISPOT employing fluorophore-labeled detection reagents). Briefly, the membrane of 96-well IPFL-bottomed microplates (Millipore) was pre-wetted for 30 seconds with 50 μL of 35% ethanol. Ethanol was then removed and each well was washed 3 times with sterile PBS. Microplates were then coated by adding 100 μL/well of a rat anti-mouse IFN-γ, a rat anti-mouse IL-5 or a rat anti-mouse IL-17 antibody solution at 10 μg/mL (PharMigen), and were incubated overnight at +4° C. On the following day, plates were washed 3 times with sterile PBS and then blocked for 2 h at +37° C. with 200 μL of RPMI GSPγ 10% FBS. After plate washing, 106 freshly isolated splenocytes/well were incubated with the pertussis antigens (PT 2.5 μg/ml; PRN 5 μg/ml; FIM 5 μg/ml; FHA 5 μg/mL) or concanavalin A (Con A, 2.5 μg/mL) as a positive control, in presence of murine IL-2 (10 U/mL). After incubation for 24 hours for IFNγ and IL-17, and 48 hours for IL-5, the plates were washed 6 times with PBS supplemented with 0.05% BSA (200 μL/well). After washing, 100 μL/well of the biotinylated anti-mouse IFN-γ (2 μg/mL) or anti-mouse IL-5 (1 μg/mL) or anti-mouse IL-17 (1 μg/mL) antibodies were added for 2 hours at room temperature in the dark. Then, the plates were washed 3 times with PBS-BSA 0.05% (200 μL/well). Then, 100 μL/well of streptavidin-PE at 1 μg/mL in PBS-BSA 0.05% was incubated for 1 hour at room temperature in dark. The plates were further washed 6 times with PBS-BSA 0.05% (200 μL/well). The plates were stored at +5° C.±3° C. in the dark until reading. Each spot, corresponding to an IFN-γ, IL-5 or IL-17 secreting cell was enumerated with an automatic ELISPOT fluorescent plate reader (Microvision). Results were expressed as number of IFN-γ, IL-5 or IL-17 secreting cells per 106 splenocytes. The geometric mean and standard deviation were calculated for each group.

Antibody Quantification

Serum IgG1 and IgG2a antibodies specific to pertussis antigens (FIM, PT, FHA, PRN), diphtheria toxoid and tetanus toxoid were titrated in a multiplex U-PLEX assay (Meso-Scale Diagnostics, Rockville, Md.).

The U-PLEX assay consists of 5 unique U-PLEX linkers that specifically bind to 5 individual spots on a 96-well U-PLEX plate. The biotin-based capture coupling mechanism involves a two-step process: (1) a linker is bound to a biotinylated antigen and (2) the linker-coupled antigen is bound to the plate. The serial dilution of serum sample, control and reference serum is added, a wash step performed, and the IgG1 or IgG2a antibodies bind to coated antigen were detected using a SULFO-TAG™ labeled ant-IgG1 or SULFO-TAG™ labeled ant-IgG2a.

### Example 1

Comparison of Genetically-Detoxified (gdPT) Immunogenicity with Chemically-Detoxified PT (PTxd)

A study was performed to compare the immunogenicity of genetically-detoxified pertussis toxin (gdPT) to that of chemically-detoxified PT (PTxd). Groups of naïve CD1 mice received two immunizations, each three weeks apart (at days 0 and 21), of gdPT or PTxd at 2.5, 0.5, 0.1 or 0.02 μg per mouse dose. In addition, to evaluating the ability of gdPT to boost a response primed by PTxd, one group of mice was immunized with a single 0.5 μg dose of PTxd, followed by two gdPT immunizations at 0.5 μg per dose, each 3 weeks apart (at days 0, 21, and 42). All formulations contained 0.066 mg AlPO₄ in 100 pt injection volume. Blood samples were collected 17 days after the second immunization (Day 38) and 8 days after the third immunization (Day 50) for analysis of IgG1 and IgG2a titers by a pertussis toxin specific ELISA and PT neutralization antibody titers.

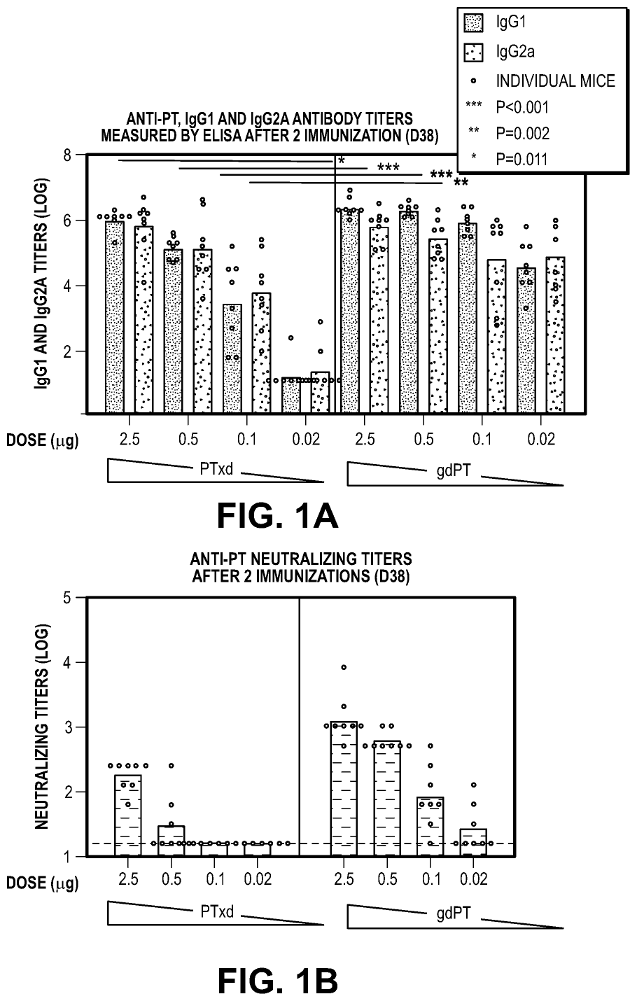

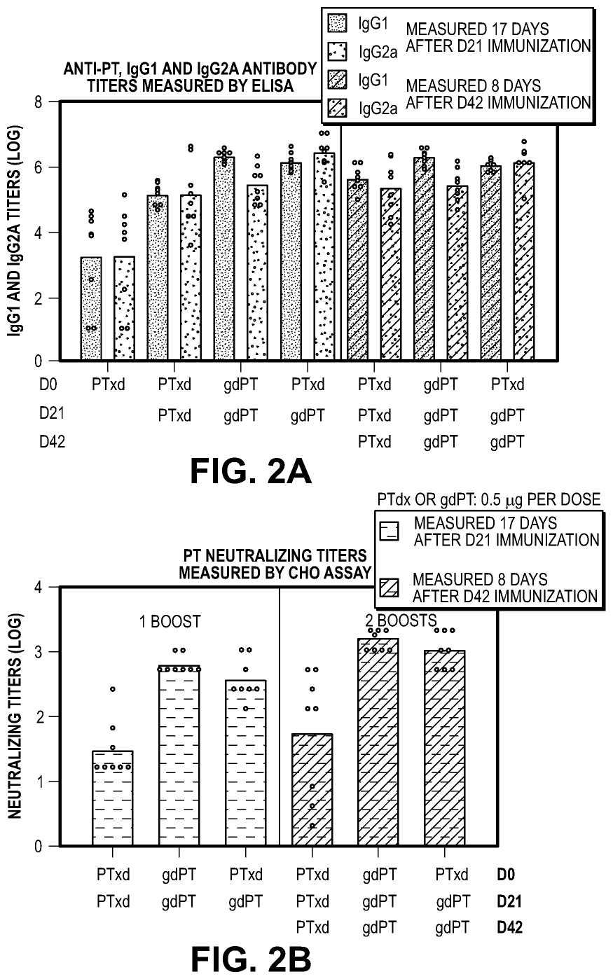

The formulations containing gdPT induced stronger and dose-dependent anti-PT specific IgG1 and IgG2a responses, particularly at lower doses, as well as higher PT neutralizing antibody titers than formulations of PTxd (FIGS. 1A-B). Both gdPT and PTxd were able to similarly boost a PT-specific IgG1 and IgG2a response primed by PTxd (FIG. 2A). However, in mice that were primed with PTxd, boosting with gdPT resulted in higher PT neutralizing antibody titers than boosting with PTxd (FIG. 2B).

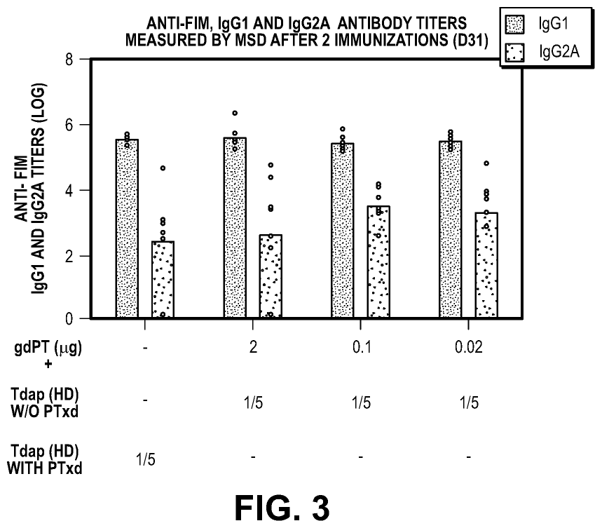

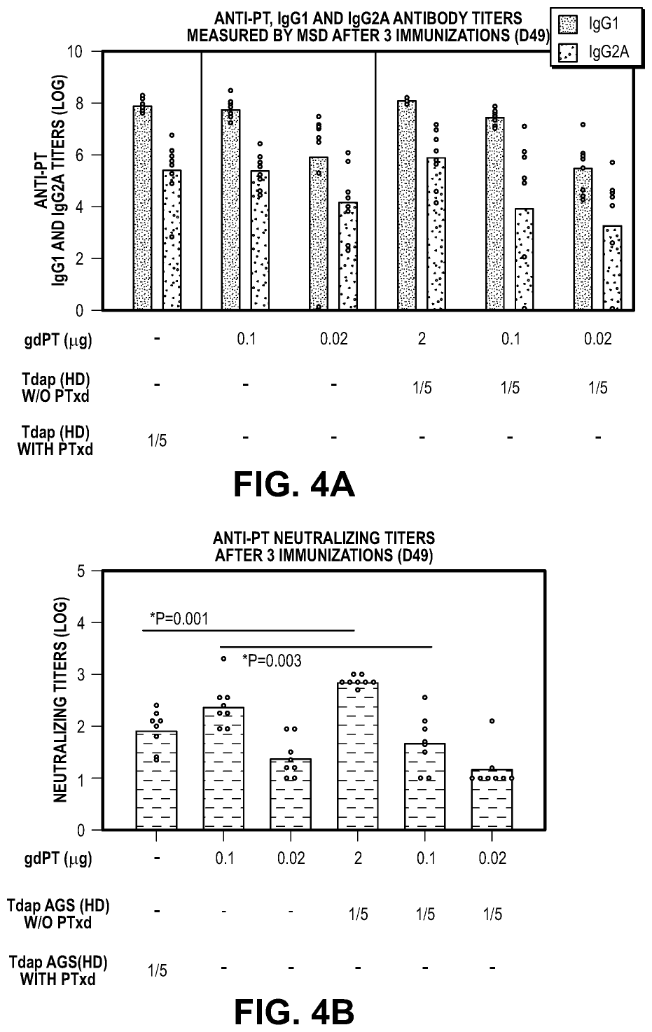

Next, a study was performed to assess whether substitution of gdPT for PTxd in the Tdap vaccine could have an effect on the immunogenicity of the other vaccine components. To address this possibility, immunogenicity of a control Tdap vaccine formulation containing PTxd was compared with that of a vaccine formulation containing gdPT. Vaccines containing gdPT at 2.0, 0.1 or 0.02 μg/dose were formulated in combination with the other Tdap vaccine antigens (1 Lf tetanus toxoid, 0.4 Lf diphtheria toxoid, 1 μg FHA, 1 μg PRN, 1.5 μg FIM2,3 and 66 μg aluminum (A100H) per mouse dose in 100 μL, or ⅕ Human Dose). A control Tdap vaccine containing 2 μg of chemically-detoxified PT was formulated with the same Tdap antigen concentrations, as a comparator. This study also included groups of mice that received gdPT alone at 0.1 or 0.02 μg per dose in A100H. CD1 mice received three immunizations of vaccine formulation, three weeks apart and were sacrificed 7 days after the last immunization. Blood samples were collected 10 days after the second immunization (FIG. 3) and at the sacrifice for analysis of PT-specific IgG1 and IgG2a titers and the PT neutralization antibody response (FIGS. 4A-B).

A modified Tdap vaccine containing gdPT (at 2 μg per dose and 3 immunizations per mouse) elicited higher PT neutralizing antibody titers than an otherwise identical control Tdap vaccine containing 2 μg per dose PTxd (FIG. 4B), although the gdPT-containing and PTxd-containing Tdap vaccines had comparable PT-specific IgG1 and IgG2a titers (FIG. 4A). Moreover, the IgG1 and IgG2a antibody titers against FIM were comparable between the gdPT-containing Tdap formulation and the PTxd-containing Tdap formulation (FIG. 3). Similar IgG1 and IgG2a profiles were observed for other vaccine antigens (i.e., tetanus toxoid, diphtheria toxoid, FHA, and PRN). Thus, gdPT did not impact the antibody responses induced by other vaccine antigens (FIG. 3). However, the presence of other Tdap vaccine antigens appeared to reduce the PT neutralizing antibody titers induced by gdPT at low doses (FIG. 4A, B). Specifically, the mean logarithmic PT neutralization titer induced by 0.1 μg gdPT 7 days after the third immunization was significantly higher (0.7-fold) in the absence of other Tdap antigens than with the Tdap antigens (FIG. 4B) although no effect was observed on the PT-specific IgG1 and IgG2a titers (FIG. 4A).

In conclusion, gdPT was confirmed to be more immunogenic than PTdx in the Tdap formulation. Also, gdPT does not interfere with the immunogenicity of other Tdap antigens in the vaccine formulation.

### Example 2: TLR Adjuvants Re-Orient a DTaP-Induced Th2 Immune Memory Response Towards Th1 Using a Long Prime-Boost Schedule

In order to test the ability of a modified Tdap formulation to boost and repolarize a previously established, Th2-biased memory response, a long immunization schedule was applied. In this study, groups of 8 CD1 mice were primed by intramuscular injection of DTaP vaccine to establish a Th2-biased memory response. In a first study, mice were boosted 6 weeks later (Day 42) with the modified Tdap (gdPT+E6020-A100H) formulation or the modified Tdap (gdPT+CpG1018-A100H) formulations. In a second study, mice received two doses of the modified Tdap booster vaccine, the first dose at 6 weeks (Day 42) and the second dose at 12 weeks (Day 84). The potential of the modified Tdap boosting formulations to re-orient the immune memory response towards Th1 was evaluated by measuring ex vivo splenic cytokine producing cells or cytokine production in supernatant after in vitro antigen re-stimulation. Antibody titers (IgG1 and IgG2a) to tetanus toxoid (TT), diphtheria toxoid (DT), PT, FHA, PRN and FIM were also determined in sera collected 1, 3 and 6 weeks after the last immunization. As controls, one group of mice was immunized with DTwP vaccine (prime) followed by DTwP vaccine (boost) and another group with DTaP vaccine (prime) followed by a boost with a control modified Tdap vaccine formulation having gdPT-A100H (20 μg/ml gdPT) but without a TLR agonist, i.e., control modified Tdap (gdPT-A100H).

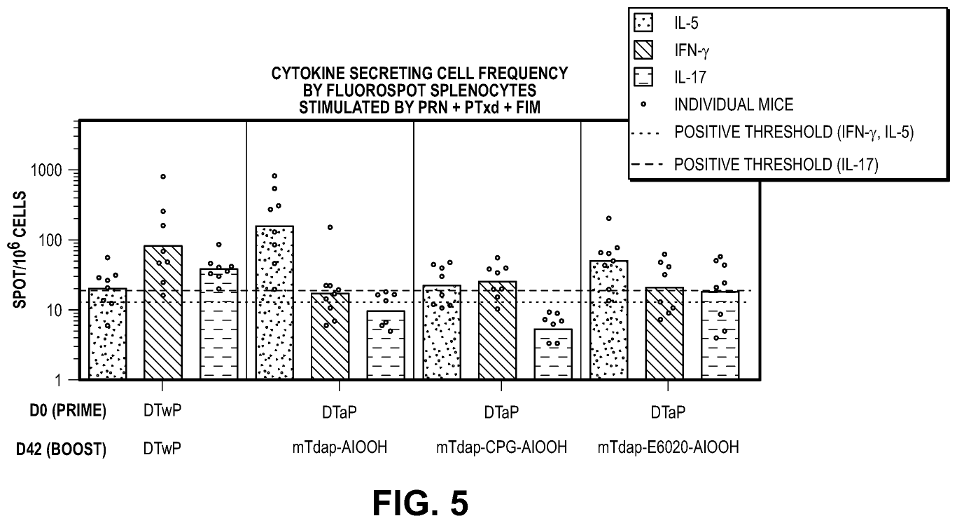

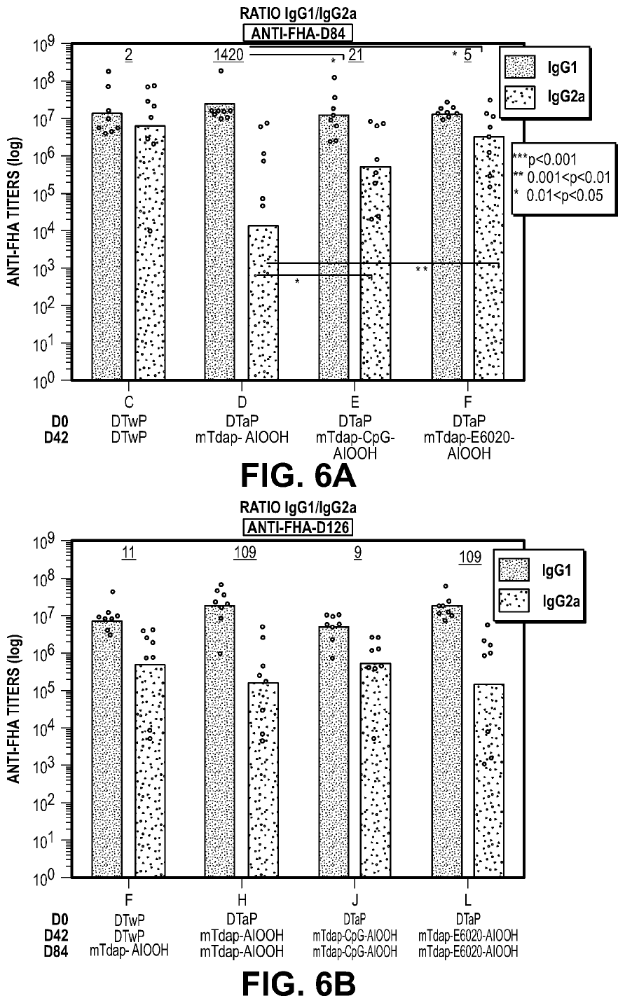

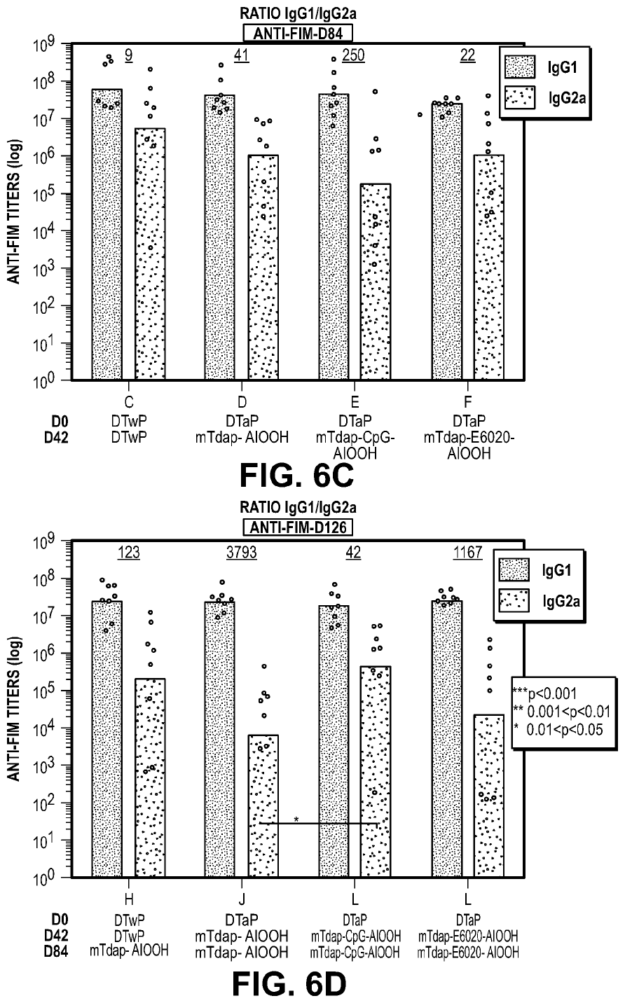

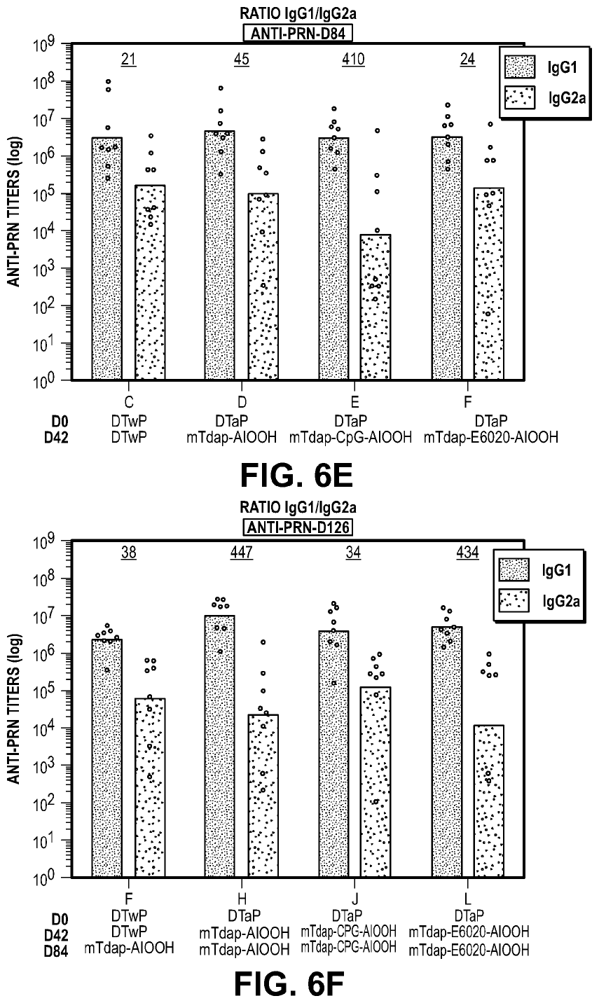

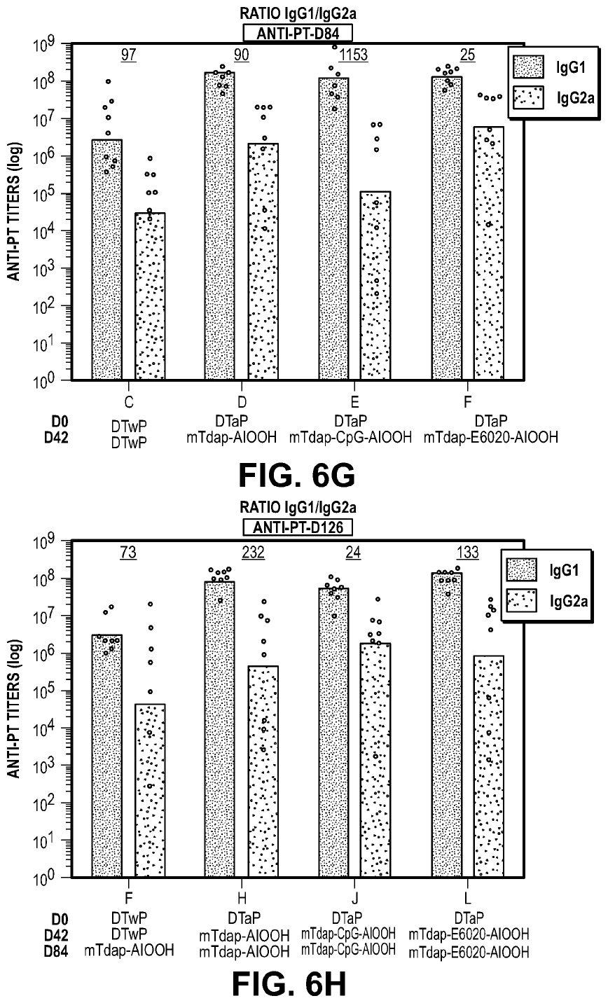

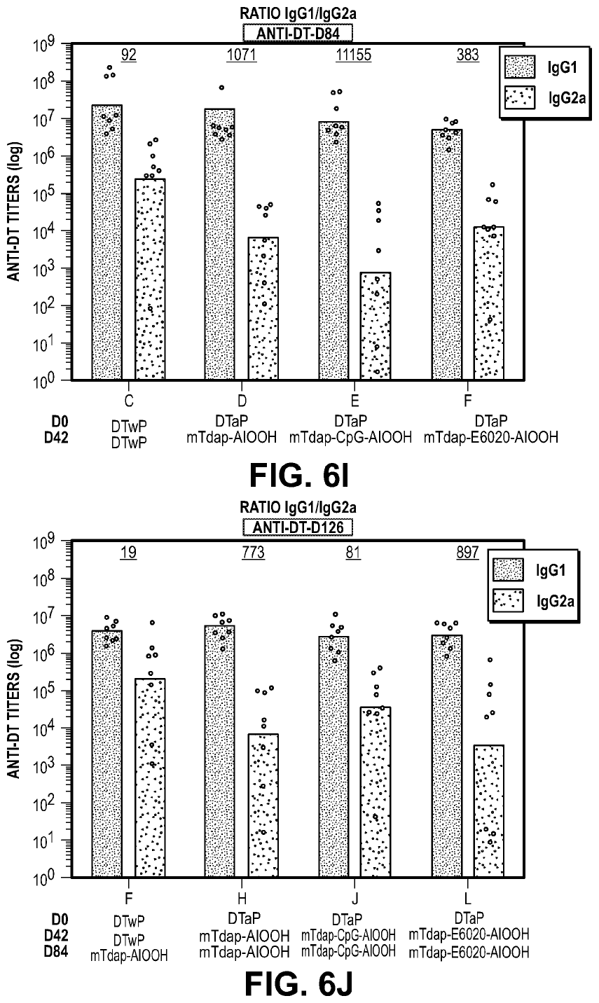

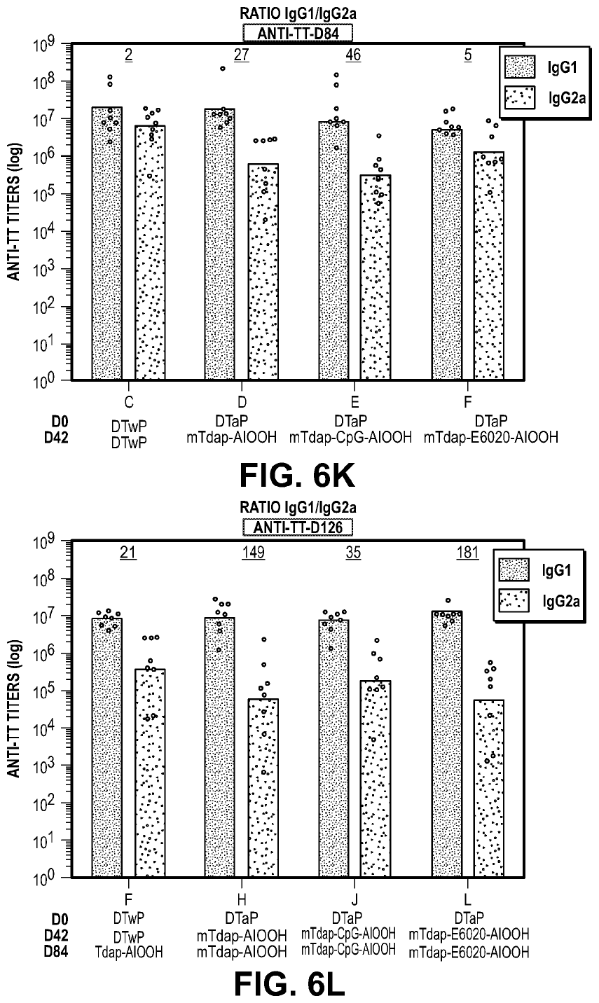

The DTwP/DTwP (prime/boost) schedule induced a less Th2-biased immune profile compared to DTaP/modified control Tdap (gdPT-A100H) (prime/boost) schedule, as evidenced by weaker IL-5 secretion (FIG. 5) and a lower IgG1/IgG2a ratio (FIGS. 6A-L). The modified Tdap (gdPT+E6020-A100H) or modified Tdap (gdPT+CpG101-A100H) boost formulations induced a significantly decreased IL-5 production as compared to the control modified Tdap (gdPT-A100H) boost vaccine (FIG. 5). However, neither of these new, modified Tdap formulations with TLR agonist adjuvants altered the IFN-γ level that was observed with the control modified Tdap (gdPT-A100H) (FIG. 5). In agreement with the overall cytokine balance towards a less Th2-biased response, a lower IgG1/IgG2a ratio was observed in mice boosted by the modified Tdap (gdPT+E6020-A100H) or modified Tdap (gdPT+CpG1018-A100H) vaccines (FIGS. 6A-L). After immunization with mTdap-CpG-A100H, a statistically significant decrease of IgG1/IgG2a ratios were observed for anti-FHA after one boost and for anti-FIM after two boosts as compared to mice immunized with mTdap-A100H, which did not contain a TLR agonist (FIGS. 6A and 6D). After immunization with mTdap-E6020-A100H, a statistically significant decrease of IgG1/IgG2a ratios for anti-FHA after one boost was observed as compared to mice immunized with mTdap-A100H, which did not contain a TLR agonist (FIG. 6A). In DTaP-primed mice, no statistically significant difference was observed for IL-17 secretion after boost by modified Tdap (gdPT+E6020-A100H) or modified Tdap (gdPT+CpG1018-A100H) formulations as compared to the control modified Tdap (gdPT-A100H) boost vaccine without TLR agonist (FIG. 5).

In conclusion, the modified Tdap (gdPT+E6020-A100H) and modified Tdap (gdPT+CpG1018-A100H) vaccines were able to alter the balance of T helper cell response towards a less Th2-biased response in DTaP primed mice, thus achieving T helper response repolarization and a shift in the Th1/Th2 balance.

### Example 3: Adoptive Transfer Model of Pertussis Immunity

The modified Tdap (gdPT+E6020-A100H) formulation and the modified Tdap (gdPT+CpG1018-A100H) formulation were tested for their ability to reactivate a DTaP-induced immune memory response for Bordetella pertussis protection in the absence of circulating antibodies induced by DTaP vaccination.

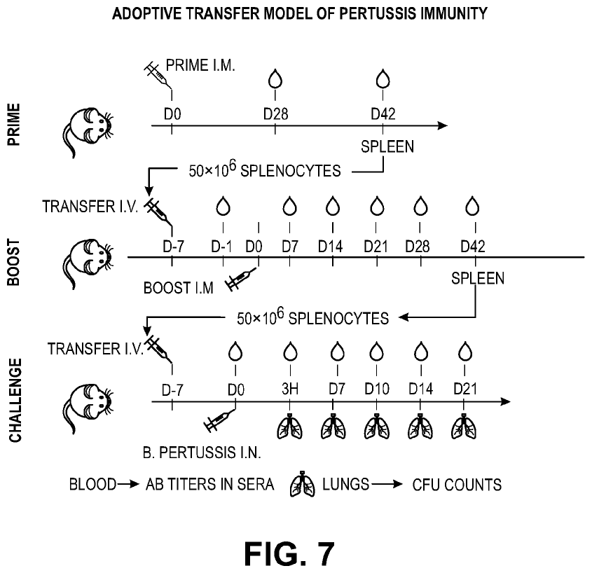

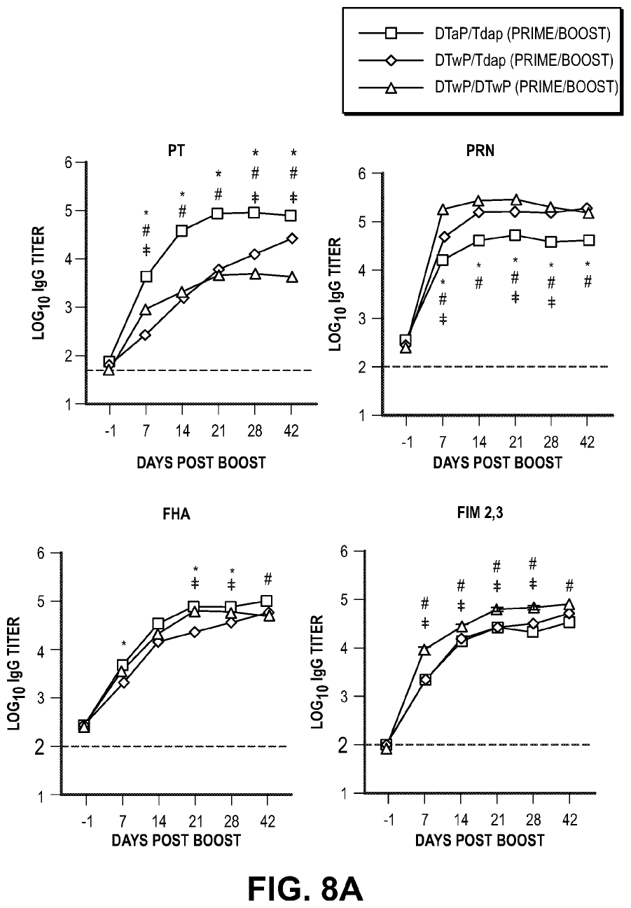

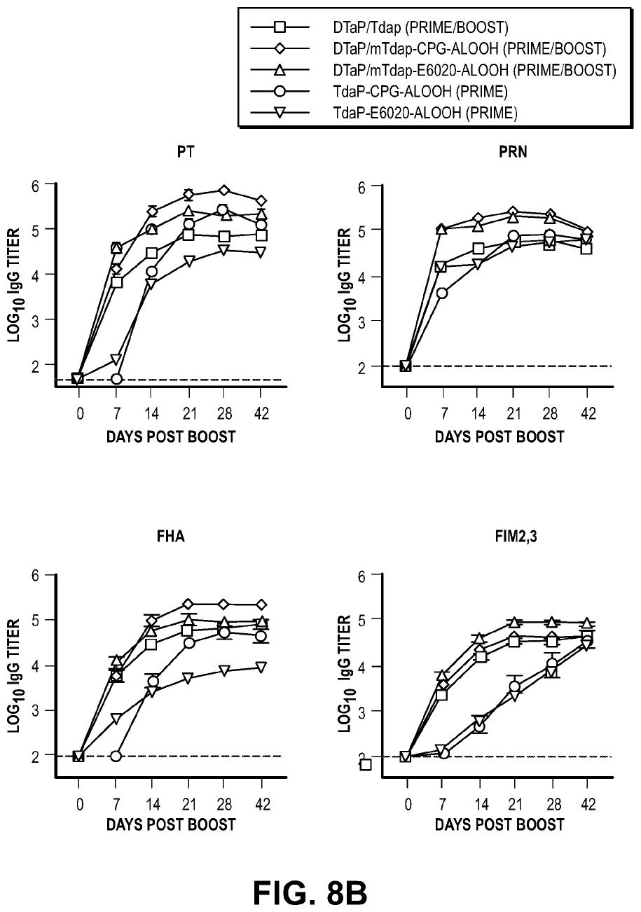

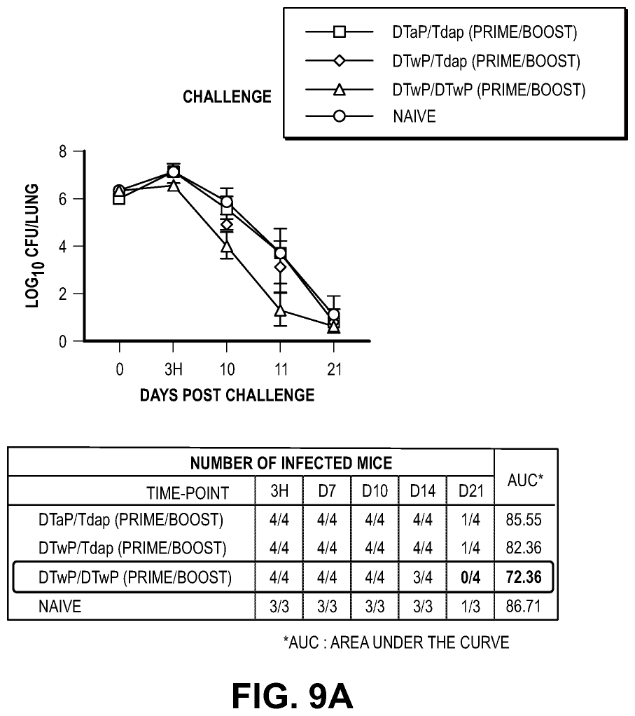

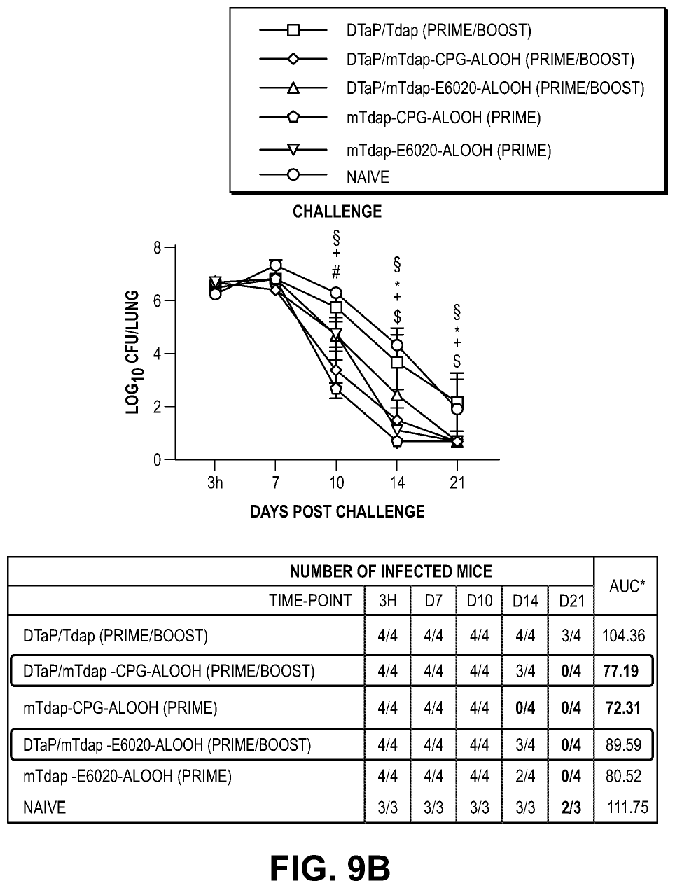

One discrepancy between humans and mice is the longevity of serum antibodies induced by DTaP priming. While these antibodies wane rapidly in humans, prolonged antibody persistence in mice may interfere with the readout of booster responses and therefore the evaluation of modified Tdap booster formulations. To address this discrepancy, a murine double transfer model was used to eliminate circulating antibodies induced by DTaP vaccination (Gavillet et al 2015 Vaccine), as summarized in FIG. 7. In this setting, adult BALB/c mice were primed once with DTwP or DTaP and their splenocytes harvested six weeks later and transferred (50×10⁶ splenocytes) to recipient, naïve BALB/c mice. Recipient mice were boosted by DTwP (only on DTwP-priming background), Tdap, the modified Tdap (gdPT+E6020-A100H) formulation or the modified Tdap (gdPT+CpG1018-A100H) formulation. Six weeks later, the splenocytes of the boosted mice were harvested and transferred (50×10⁶ splenocytes) to new recipient, naïve BALB/c mice. One week after the transfer, the double adoptive transfer recipient mice were challenged with 10⁶ live Bordetella pertussis and sacrificed on day 0, 7, 10, 14 or 21 after challenge. Bacterial counts in the lungs of the sacrificed mice were measured as an indication of effective immune recall responses in the absence of circulating antigen-specific antibodies induced by vaccination (FIG. 9A-B). Vaccine induced responses were also monitored by detection of PT-, PRN-, FHA- and FIM2,3-specific IgG antibody responses in sera collected at 7, 14, 21, 28, and 42 days after the boost (FIG. 8A-B). Accelerated and higher vaccine antigen IgG titers are characteristics of a recall antibody response in adoptive transferred mice.

In the absence of circulating antibodies at the time of B. pertussis challenge, memory cells in DTwP/DTwP (prime/boost) mice provided early B. pertussis control and facilitated bacteria clearance from the lungs (FIG. 9A), due at least partially to rapid generation of antibodies specific to pertussis vaccine antigens, such as PT and PRN (FIG. 8A). By contrast, DTaP priming/Tdap boost did not accelerate bacterial clearance in the lungs as compared to the naïve control animals (FIG. 9A-B). In this adoptive transfer model, mice primed with DTaP and boosted with modified Tdap (gdPT+CpG1018-A100H) and modified Tdap (gdPT+E6020-A100H) boost showed accelerated and higher anti-PT, FHA, FIM IgG titers as compared to mice receiving a Tdap boost (FIG. 8B).

Replacing chemically-detoxified PT by gdPT in the Tdap boost vaccine had no impact on B. pertussis lung clearance despite an enhanced PT-specific IgG antibody response (data not shown). A strong reactivation of antigen-specific memory response was nevertheless observed for the modified Tdap (gdPT+CpG1018-A100H) vaccine. Early bacterial control and accelerated bacterial clearance was recorded in the DTaP priming/modified Tdap (gdPT+CpG1018-A100H) boost group with similar kinetics as the DTwP priming/DTwP boost group (FIG. 9A-B). Similarly, rapid generation of antibodies specific to PT and FHA vaccine antigens was observed with the modified Tdap (gdPT+CpG1018-A100H) formulation (FIG. 8B). Testing the modified Tdap (gdPT+E6020-A100H) suggested early reactivation of pertussis specific antibody responses against some aP antigens, e.g., PT and FHA (FIG. 8B). However, less impact was observed by the modified Tdap (gdPT+E6020-A100H) boost on early B. pertussis control, as compared to the modified Tdap (gdPT+CpG1018-A100H) boost (FIG. 9B). In conclusion, this adoptive transfer experiment in mice demonstrated that the modified Tdap (gdPT+E6020-A100H) and modified Tdap (gdPT+CpG1018-A100H) booster formulations are able to improve the pertussis-specific recall antibody responses resulting in accelerated protection against bacteria colonization.

Materials and Methods for Example 3

Mice: Adult BALB/cByJ mice were purchased from Charles River (L'Arbresle, France) and kept under specific pathogen free conditions. Mice were used at 6-8 weeks of age. All animal experiments were carried out in accordance with Swiss and European guidelines and approved by the Geneva Veterinary Office.

Adoptive transfer: Spleens were harvested 42 days after prime or boost. Single cell suspensions were obtained by mechanical disruption of the organs and further processed for red blood cell lysis. 50×10⁶ splenocytes were transferred intravenously in a volume of 100 μl into naïve recipient mice.

B. pertussis challenge: Streptomycin-resistant Bordetella pertussis Tahoma I derivative BPSM were grown on Bordet-Gengou agar (Difco) supplemented with 1% glycerol, 10% defibrinated sheep blood (Chemie Brunschwig AG) and 100 μg/ml streptomycin. After 24 h at 37° C., colonies were transferred into PBS and the optical density of the bacterial suspension measured. 1×10⁶ colony-forming units (CFU) were instilled intranasally in a volume of 20 μl into mice anesthetized by intraperitoneal injection of Ketasol (100 mg/kg; Graeub) and Rompun (10 mg/kg; Bayer). Mice were sacrificed 3 hours after infection for quantification of the initial numbers of viable B. pertussis CFUs in the lungs and at days 7, 10, 14 and 21 for determination of bacterial colonization. Briefly, lungs homogenates were plated onto Bordet-Gengou agar plates and the number of CFUs was counted after 4 days of incubation at 37° C. The measure of protective efficacy was expressed as a ratio of the area under the clearance curve (AUC) between naïve control and immunized mice.

ELISA: PT-, PRN-, FHA- and FIM2,3-specific antibody titers were determined by Ag-capture ELISAs in serum samples collected at indicated time-points. Briefly, 96-well plates (Nunc MaxiSorp™; Thermo Fisher Scientific) were coated with PT (1 μg/ml), PRN (5 μg/ml), FHA (5 μg/ml) or FIM2,3 (2 μg/ml) in carbonate buffer, pH 9.6, overnight at 4° C. After saturation with PBS, 0.05% Tween 20, and 1% BSA (Sigma) for 1 h at 37° C., wells were incubated with 2-fold serial dilutions of individual or pooled mouse sera for 1 h at 37° C. Secondary horseradish peroxidase (HRP) conjugated anti-mouse IgG, anti-mouse IgG2a (both from Invitrogen), and anti-IgG1 (BD Pharmingen) were incubated for 1 h at 37° C. and revealed with 2,2′-Azinobis [3-ethylbenzthiazoline-6-sulfonic acid]-diammonium salt (ABTS) substrate for 1 h. The optical density of each well was measured at 405 nm and the data analyzed with SoftMax Pro software. Results in FIG. 8 for IgG and IgG1 are expressed as Log₁₀ of Ag-specific titers determined in reference to serial dilutions of a WHO/NIBSC reference reagent Bordetella pertussis anti-serum (NIBSC code: 97/642) and IgG2a in reference to serial dilutions of a titrated pool of hyperimmune sera from immunized mice.

Statistical analysis: Values are expressed as mean±SEM. Statistical analysis were performed using unpaired t-test or one-way ANOVA followed by a Tukey multiple comparison test when more than 2 groups of mice were tested. All analysis were done using the GraphPrism software. Differences with p>0.05 were considered insignificant.

### Example 4: Mouse Intranasal Challenge Model

A mouse lung clearance model that reflects the clinical efficacy levels of licensed pertussis vaccines has been developed, implemented, and validated (Guiso N. et al., Vaccine. 1999; 17:2366-76). The mouse Intranasal Challenge Assay (INCA) model is recommended by the WHO for non-clinical testing for registration of new vaccine candidates and known as a valid research model in vaccine research and development (World Health Organization. WHO Expert Committee on Biological Standardization. Recommendations to Assure the Quality Safety and Efficacy of Acellular Pertussis Vaccines. 2011; WHO/BS/2011.2158.) Moreover, the WHO advisory group has agreed that the INCA is useful for assessing potential impacts of changing formulation and/or manufacturing process for pertussis vaccines.

In this model, following intranasal challenge with a sublethal dose, bacteria adhere to ciliated epithelium in the trachea and invade macrophages in the lungs. The disease persists, lasting 4-8 weeks with leukocytosis and immune suppression. A rapid clearance of the infection is observed following re-challenge of convalescent mice. The INCA model was shown to discriminate among acellular pertussis vaccines that showed different efficacies in clinical trials (Guiso N. et al 1999).

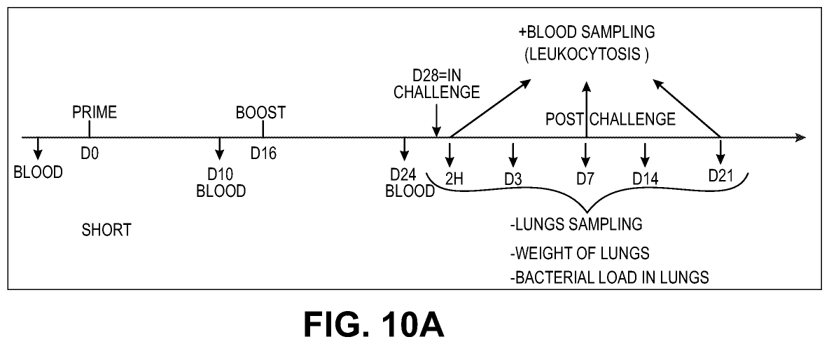

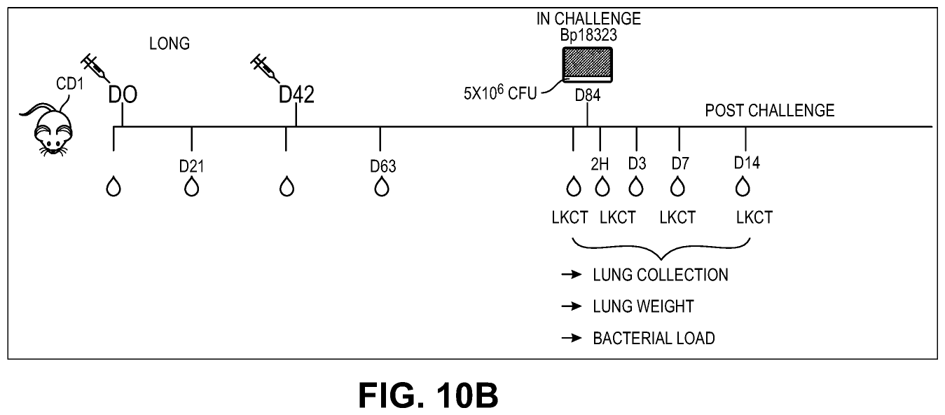

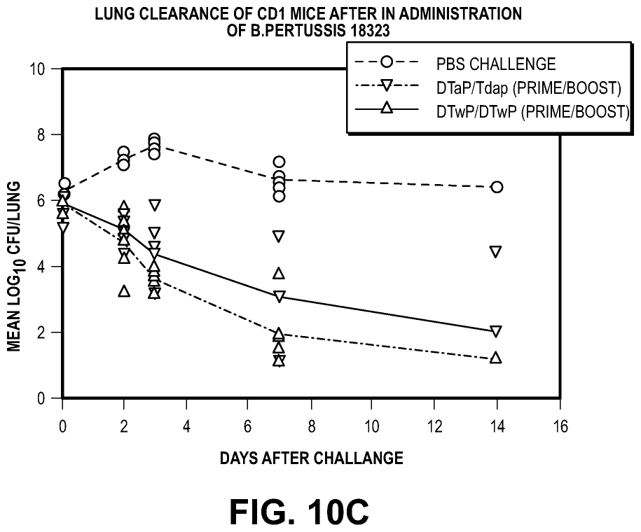

The INCA mouse model was used to evaluate whether new modified Tdap booster vaccines, such as the modified Tdap (gdPT+CpG1018-A100H) and the modified Tdap (gdPT+E6020-A100H) vaccines will elicit a short-term level of protection equal to or higher than a Tdap control boost vaccine in mice primed with DTaP. FIG. 10A shows a pictorial representation of a mouse Intranasal Challenge Assay (INCA) using a short schedule, and FIG. 10B shows a pictorial representation of a mouse INCA using a long Schedule. FIG. 10C shows that a DTwP/DTwP (prime/boost) schedule and a DTaP/Tdap (prime/boost) schedule protect mice against Bordetella pertussis lung colonization following intranasal challenge in this model using a short schedule.

Mouse Intranasal Challenge Assay Using Short Prime-Boost Schedule

A 2-week interval short immunization schedule was applied as described in the WHO harmonized protocol (WHO Expert Committee on Biological Standardization, 2011). The readouts for this study were the reduction of bacterial load in the lungs (CFU/lung) as well as hyperleukocytosis based on White Blood Cell (WCB) count in the blood.

Groups of 40 mice were injected with ⅕ human dose of DTaP by intramuscular route and received a second injection two weeks later with Tdap, the modified Tdap (gdPT+E6020-A100H) vaccine, or with a control Tdap vaccine formulated with chemically-detoxified PT, A100H, and without a TLR agonist (control Tdap (PTxd-A100H)). A group that received two injections of DTwP (prime/boost) was also included. Two control groups received either PBS (infection control), or E6020-A100H adjuvant alone to verify that aluminum salts and E6020 had no effect on colonization compared to the infection control.

Two weeks following the second immunization, mice were challenged by instillation of a suspension of B. pertussis 18323 into the nostrils. Following challenge, eight mice from each group were sacrificed at 2 hours post-challenge, and at 3, 7, 14 and 21 days post-challenge. Lungs were removed aseptically and homogenized individually to measure bacterial load. The mean CFU per lung was determined by counting the colonies grown on Bordet-Gengou agar plates after plating serial dilutions of the homogenate. Blood samples were also collected following challenge and WBC counts were performed.

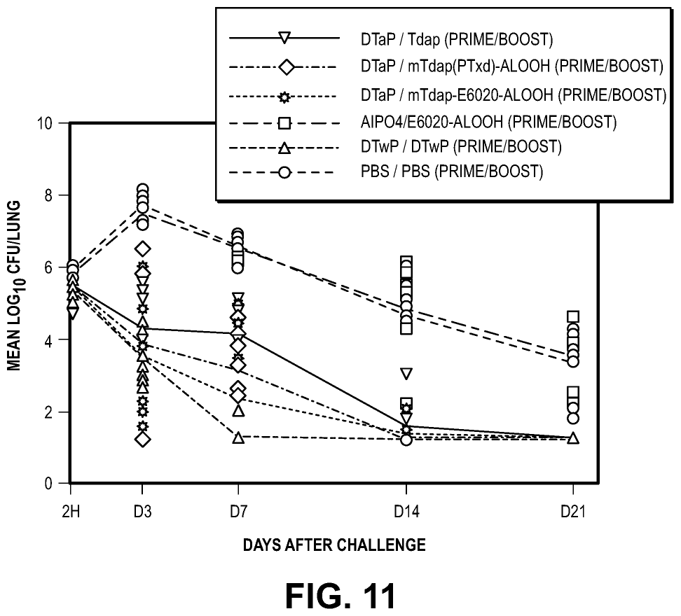

As shown in FIG. 11, B. pertussis 18323 colonized and expanded in the lung of PBS control mice three days after the challenge before entering a clearance phase. Complete B. pertussis lung clearance took more than 21 days in the PBS control mice. There was no impact of the adjuvant alone on lung colonization and disease. Reduction in B. pertussis lung colonization was observed in all vaccinated groups 3 days post challenge with baseline bacterial load reached at 14 days post challenge for all formulations. DTwP caused the fastest B. pertussis lung clearance with baseline reached at 7 days post challenge. The results showed that the modified Tdap (gdPT+E6020-A100H) formulation drastically reduced the bacterial load in the lungs when compared to the infection control (4 log at day 3 and 5 log at day 7) demonstrating its capacity to prevent colonization of the lower respiratory tract following intranasal challenge with B. pertussis 18323. Notably, the clearance profile was closer to that obtained for DTwP/DTwP (prime/boost) and farther from that obtained for DTaP/Tdap (prime/boost). The difference in bacterial load obtained at day 7 between the modified Tdap (gdPT+E6020-A100H) formulation boost and Tdap (containing chemically-detoxified PT) boost was significant (p value<0.0001). All vaccines were protective against disease (FIG. 11) and prevented hyperleukocytosis. This study demonstrated that the modified Tdap (gdPT+E6020-A100H) formulation significantly accelerates Bordetella pertussis clearance as compared to Tdap (ADACEL®) in the mouse INCA using a short prime-boost schedule.

Mouse Intranasal Challenge Assay Using Long Prime-Boost Schedule

In order to confirm the efficacy of the new, modified Tdap formulations in mice that have a previously established aP-primed immunological background, a study was conducted to evaluate the capacity of the modified Tdap (gdPT+E6020-A100H) booster to prevent B. pertussis lung colonization and disease in mice using a long prime-boost schedule, as discussed above. See FIG. 10B. In this study, the acellular pertussis vaccine control was a Tdap vaccine (control Tdap (PTxd-A100H)) containing chemically-detoxified PT but modified to contain the same doses of antigens and aluminum salt (A100H) as in the modified Tdap (gdPT+E6020-A100H) formulation to better appreciate the role of gdPT and TLR agonist in the new modified Tdap formulation.

Groups of 40 mice were injected with ⅕ human dose of DTaP by intramuscular route and received a second injection six weeks later with the modified Tdap (gdPT+E6020-A100H) or with the control Tdap (PTxd-A100H). A group primed with DTwP and boosted with DTwP was also included. Six weeks following the second immunization, mice were challenged by instillation of a suspension of B. pertussis 18323 into the nostrils. Following challenge, eight mice from each group were sacrificed at 2 hours post-challenge, and at 1, 2, 3, 7, and 14 days post-challenge. Lungs were removed aseptically and homogenized individually to measure bacterial load. The mean CFU per lung was determined by counting the colonies grown on Bordet-Gengou agar plates after plating serial dilutions of the homogenate. Blood samples were also collected following challenge and WBC counts were performed.

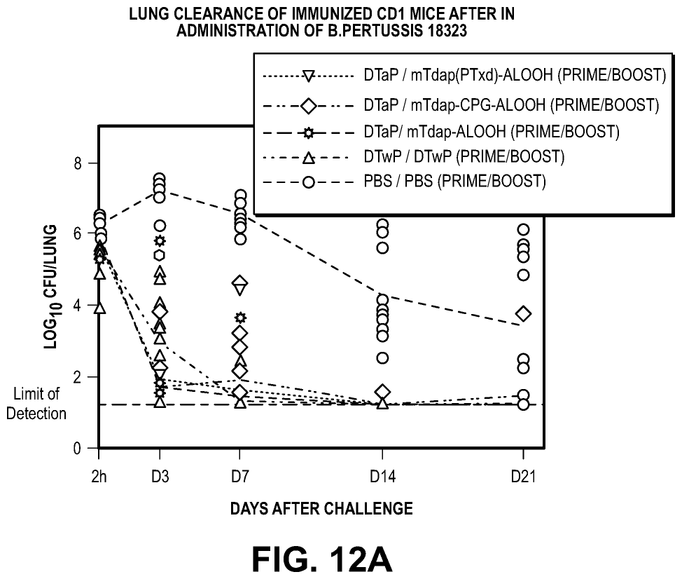

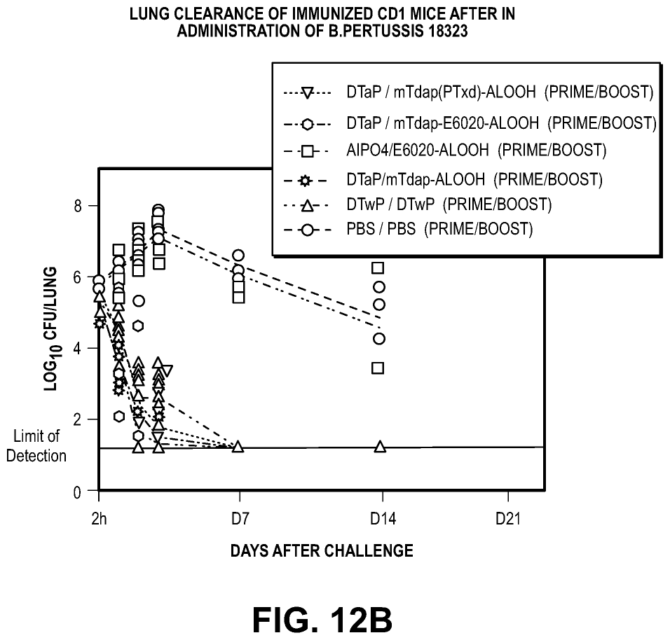

As shown in FIG. 12B, the modified Tdap (gdPT+E6020-A100H) formulation drastically reduced the bacterial load in the lungs (6 log reduction) as soon as day 1 when compared to the infection control. A significant difference (p value=0.007) was also observed between modified Tdap (gdPT+E6020-A100H) and the control Tdap (PTxd-A100H) at day 1 with a 7.7 fold reduction of CFU counts in lungs. The bacterial load for all vaccinated mice returned to base line by day 7, indicating no detectable bacteria in the lungs (FIG. 12B).

This study demonstrated that the modified Tdap (gdPT+E6020-A100H) formulation was more efficacious in a long prime-boost schedule for preventing lung colonization (at least during an early phase of the infection) than a Tdap control without gdPT or a TLR agonist, suggesting that this effect is due to the gdPT and/or TLR agonist contained in the modified Tdap formulation.

### Example 5: Th17 Secreting Cells Measured by Fluorospot in Splenocytes

To study the ability of a TLR adjuvant to repolarize an immune response from a Th2 to a more Th-17 response, IL-17 cytokines were measured in the following experiments. CD1 mice were primed with DTaP composition and boosted twice, 2 weeks apart on DO and D21 with mTdap-E6020-ALOOH with or without the TLR4 adjuvant in a dose-effect design. E6020 and AlOOH were present in the mTdap formulation in an amount of 10 μg and 66 μg, respectively. The mTdap antigen components were as follows per dose of 0.1 mL:

| | gdPT | FHA | FIM2/3 | PRN | D | T | TLR4 |
|---|---|---|---|---|---|---|---|
| | (μg) | (μg) | (μg) | (μg) | (Lf) | (Lf) | (μg) |
| mTdap per 0.1 | 2 | 1 | 1.5 | 1 | 0.4 | 1 | 0, 0.1, |
| mL | | | | | | | 0.5, 1, 4 |
| | | | | | | | and 10 |
| HD equivalent | 10 | 5 | 7.5 | 5 | 1 | 5 | |
| per 0.5 ml | | | | | | | |

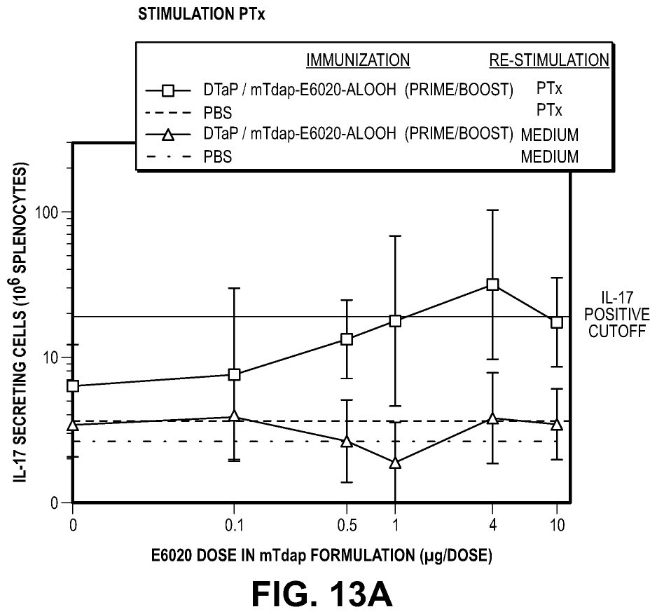

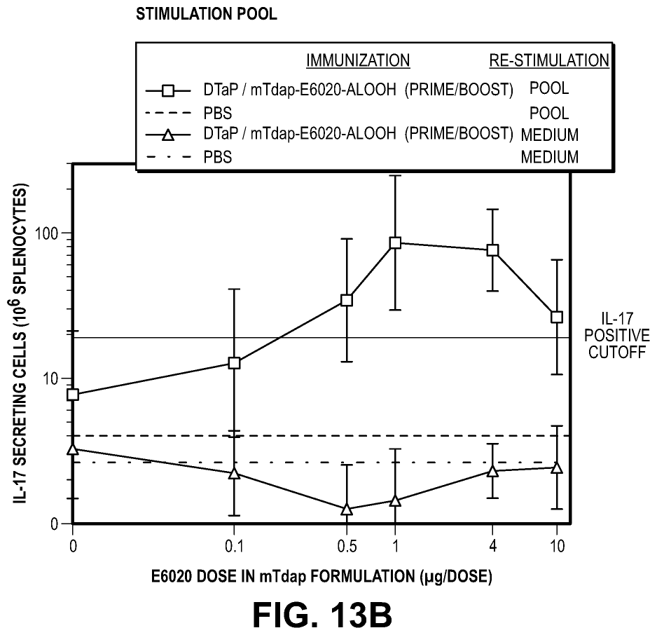

Blood sampling was collected from mice on D20 (200 μL) and D35 (exsanguination) for all groups under anesthesia (Imalgen/Rompun 80 mg/kg ketamine and 16 mg/kg xylazine, 100 μL/10 g or isoflurane). Cellular immune response in the spleen cells were analyzed by ex vivo fluorospot assay to measure the IL-17 secreting cells and by MSD assay to measure the secretion of IL-17. The fluorospot technique is described above. The production of Th17 cytokines (IL-17) was measured in the supernatant of splenocytes after 3 days of in vitro stimulation with 2.5 μg/mL of PTx (FIG. 13A) or a pool of pertussis antigens (2.5 μg/mL PTx, 5 μg/mL PRN, 5 μg/mL FIM) using the fluorospot assay (FIG. 13B) and the MSD Uplex Kit.

As shown in FIGS. 13A-B, IL-17 secreting cell frequency was below the positive cutoff in the fluorospot assay after mTdap vaccine injection without TLR4 (below the line of 19 spots/10⁶ cells). The mTdap-E6020 formulations induced increasing amounts of IL-17 secreting cells in a dose dependent manner from 0.1 μg to 4 μg/dose, when the splenocytes were re-stimulated with either PT toxoid (PTx) (FIG. 13A) or a pool of pertussis antigens (FIG. 13B). A mTdap boost vaccine containing 4 μg E6020/dose increased IL-17 secreting cell frequency above the positive cutoff in the fluorospot assay when re-stimulated with PTx (FIG. 13A), while mTdap boost vaccines containing 0.5, 1, 4, and 10 μg E6020/dose increased IL-17 secreting cell frequency above the positive cutoff in the fluorospot assay when re-stimulated with a pool of pertussis antigens (FIG. 13B).

In general, cytokine secretion profiles measured by MSD were in agreement with cytokine secreting cell frequency measured by fluorospot (data not shown). The mTdap formulations containing E6020 induced IL-17 secretion in a dose-effect manner with the highest effect reached from 0.5 μg to 4 μg/dose.

### Example 6: Thermal Stability

The nanoDSF method was performed using the Prometheus NT.48 system (Nano Temper Technologies, Munich, Germany). nanoDSF uses intrinsic fluorescence to evaluate changes in aromatic residues (fluorophores) within proteins in response to the changes in their local environment. The shift and intensity change in fluorescence emission is monitored, with a change in the intrinsic fluorescence indicating that the protein has unfolded. Thermal stability of protein is characterized using the melting temperature (Tm), which indicates the point at which half the protein is unfolded. In the nanoDSF method, this is determined by using the ratio of fluorescence recorded at 330 nm and 350 nm. This ratio has shown to be more sensitive in detecting Tm as compared to the use of a single wavelength. Samples were filled in capillary tubes without any further preparation and excited at 285 nm with 100% power output. The thermal profiles were recorded from 20 to 95° C. with 2° C./min scan rate.

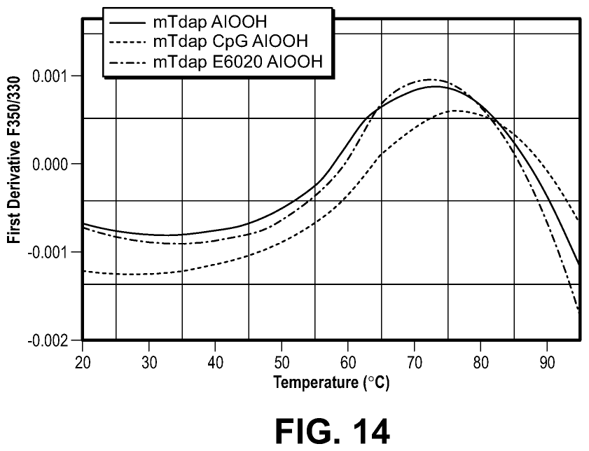

The tertiary structure and thermal stability of different vaccine formulations can be assessed by nanoDSF, to asses if any difference in conformation can be detected when the vaccines are formulated with different adjuvants. For all vaccine formulations one thermal transition was detected. FIG. 14. When the mTdap vaccine (gdPT) was adsorbed to AlOOH (mTdap-A100H) it had a thermal transition of 74.6° C. Similarly, when the mTdap-A100H formulation contains E6020 (mTdap E6020-A100H), the thermal transition was 74.2° C. When the mTdap-A100H formulation contains CpG (mTdap CpG-A100H) there was small increase in thermal transition, now detected at 77.0° C.

While one or more exemplary embodiments have been described in the specification, it will be understood by those of ordinary skill in the art that various changes in form and details may be made therein without departing from the spirit and scope of the inventive concept as defined by the following claims.

1. An acellular pertussis (aP) booster vaccine, comprising a tetanus toxoid, a diphtheria toxoid, a detoxified pertussis toxin, filamentous hemagglutinin, pertactin, fimbriae types 2 and 3, at least one toll-like receptor (TLR) agonist, and an aluminum salt, wherein the at least one TLR agonist is formulated with the aluminum salt.

2. The aP booster vaccine of claim 1, wherein the TLR agonist is a TLR4 agonist and/or a TLR9 agonist.

3. The aP booster vaccine of claim 2, wherein the TLR4 agonist comprises E6020.

4. The aP booster vaccine of claim 2, wherein the TLR9 agonist comprises CpG1018.

5. The aP booster vaccine of claim 1, wherein the tetanus toxoid is present in an amount of 8-12 Lf/mL and optionally 9-11 Lf/mL or 10 Lf/mL.

6. The aP booster vaccine of claim 1, wherein the diphtheria toxoid is present in an amount of 3-8 Lf/mL and optionally 3-6 Lf/mL or 4-5 Lf/mL.

7. The aP booster vaccine of claim 1, wherein the detoxified pertussis toxin is a genetically detoxified pertussis toxin and is present in an amount of 16-24 μg/mL and optionally 18-22 μg/mL or 20 μg/mL.

8. The aP booster vaccine of claim 1, wherein the filamentous hemagglutinin is present in an amount of 5-15 μg/mL and optionally 8-12 μg/mL or 10 μg/mL.

9. The aP booster vaccine of claim 1, wherein the pertactin is present in an amount of 5-15 μg/mL and optionally 8-12 μg/mL or 10 μg/mL.

10. The aP booster vaccine of claim 1, wherein the fimbriae types 2 and 3 are present in an amount of 10-20 μg/mL and optionally 14-16 μg/mL or 15 μg/mL.

11. The aP booster vaccine of claim 2, wherein the TLR4 agonist is present in an amount of no more than 10 μg/mL and optionally 0.5-5 μg/mL or no more than 2 μg/mL.

12. The aP booster vaccine of claim 2, wherein the TLR9 agonist is present in an amount of 250-750 μg/mL and optionally 400-600 μg/mL or 500 μg/mL.

13. The aP booster vaccine of claim 1, further comprising a tris-buffered saline.

14. The aP booster vaccine of claim 1, having an aluminum concentration of 0.5-0.75 mg/mL and optionally 0.66 mg/mL.

15. The aP booster vaccine of claim 1, wherein at least one of the tetanus toxoid, the diphtheria toxoid, and the genetically-detoxified pertussis toxin is adsorbed to the aluminum salt.

16. The aP booster vaccine of claim 1, wherein the aluminum salt is an aluminum hydroxide or an aluminum phosphate.

17. The aP booster vaccine of claim 1, wherein the tetanus toxoid is present in an amount of 9-11 Lf/mL and optionally 8-12 Lf/mL, the diphtheria toxoid is present in an amount of 3-8 Lf/mL and optionally 3-5 Lf/mL, the detoxified pertussis toxin is a genetically detoxified pertussis toxin and is present in an amount of 16-24 μg/mL and optionally 18-22 μg/mL, the filamentous hemagglutinin is present in an amount of 5-15 μg/mL and optionally 8-12 μg/mL, the pertactin is present in an amount of 5-15 μg/mL and optionally 8-12 μg/mL, the fimbriae types 2 and 3 are present in an amount of 10-20 μg/mL and optionally 14-16 μg/mL, the aluminum salt is aluminum hydroxide and is present at a concentration of 0.25-0.75 mg/mL and optionally 0.6-0.7 mg/mL, and wherein the TLR agonist is a TLR4 agonist and/or a TLR9 agonist.

18. The aP booster vaccine of claim 17, wherein the TLR4 agonist comprises E6020 and is present in an amount of no more than 2 μg/mL or wherein the TLR9 agonist comprises CpG1018 and is present in an amount of 400-600 μg/mL.

19. The aP booster vaccine of claim 18, wherein the tetanus toxoid is present in an amount of 10 Lf/mL, the diphtheria toxoid is present in an amount of 4-5 Lf/mL, the detoxified pertussis toxin is a genetically detoxified pertussis toxin and is present in an amount of 20 μg/mL, the filamentous hemagglutinin is present in an amount of 10 μg/mL, the pertactin is present in an amount of 10 μg/mL, the fimbriae types 2 and 3 are present in an amount of 15 μg/mL, the aluminum salt is aluminum hydroxide and is present at a concentration of 0.66 mg/mL, and wherein the TLR agonist is a TLR4 agonist and/or a TLR9 agonist.

20. The aP booster vaccine of claim 19, wherein the TLR4 agonist comprises E6020 and is present in an amount of 0.5-5 μg/mL or wherein the TLR9 agonist comprises CpG1018 and is present in an amount of 500 μg/mL.

21. The aP booster vaccine of claim 20, wherein the aP booster vaccine is in a 0.5 mL unit dose form for administration to a human subject and wherein the tetanus toxoid is present in an amount of 5 Lf, the diphtheria toxoid is present in an amount of 2-2.5 Lf, the genetically detoxified pertussis toxin is present in an amount of 10 μg, the filamentous hemagglutinin is present in an amount of 5 μg, the pertactin is present in an amount of 5 μg, the fimbriae types 2 and 3 are present in an amount of 7.5 μg/mL, the aluminum hydroxide is present at a concentration of 0.33 mg, and E6020 is present in an amount of 0.25-2.5 μg or CpG1018 is present in an amount of 250 μg.

22. The aP booster vaccine of claim 1, wherein the detoxified pertussis toxin is a genetically-detoxified pertussis toxin and comprises a R9K mutation and an E129G mutation.

23. The aP booster vaccine of claim 1, further comprising a Haemophilus influenzae type-b saccharide conjugate, a hepatitis B virus surface antigen and/or an inactivated polio virus.

24. A method of inducing an immune response in a human subject who has been previously exposed to B. pertussis antigen, the method comprising administering to the human subject the aP booster vaccine of claim 1, wherein the previous exposure to B. pertussis antigens induces a Th2-biased immune response in the human subject, and wherein the aP booster vaccine reorients the Th2-biased immune response towards a Th1-biased or a Th1/Th17-biased immune response in the human subject.

25. The method of claim 24, wherein the human subject has received an acellular pertussis (aP) priming vaccine prior to administering the aP booster vaccine and wherein the aP priming vaccine induces a Th2-biased immune response in the human subject.

26. The method of claim 24 or 25, wherein the human subject is 4 years of age or older when the aP booster vaccine is administered.

27. The method of claim 24 or 25, wherein the human subject is 10 years of age or older when the aP booster vaccine is administered.

28. The method of claim 24, wherein the Th1-biased immune response is characterized by one or more of decreased IL-5 production or a lower IgG1/IgG2a ratio, as compared to the Th2-biased immune response induced by the aP priming vaccine or an aP booster vaccine that does not contain a TLR agonist, and the Th1/Th17-biased response is characterized by increased IL-17 production and one or more of decreased IL-5 production or a lower IgG1/IgG2a ratio, as compared to the Th2-biased immune response induced by the aP priming vaccine or an aP booster vaccine that does not contain a TLR agonist.

29. The method of claim 24, wherein the aP priming vaccine comprises a tetanus toxoid, a diphtheria toxoid, a pertussis toxin, filamentous hemagglutinin, pertactin, and fimbriae types 2 and 3, with the proviso that the aP priming vaccine does not contain a TLR agonist.


SEQUENCE LISTING (1 sequence)

SEQ ID NO: 1 - DNA, 22 bp, Artificial Sequence
  CpG oligodeoxynucleotide (ISS1018)
tgactgtgaacgttcgagatga